SEQUENCE LISTING (22 sequences)

SEQ ID NO: 1 - DNA, 22 bp, Artificial sequence
  consensus sequence
  misc_feature (1)..(1): n is a, c, g or t
  misc_feature (5)..(6): n is a, c, g or t
  misc_feature (9)..(10): n is a, c, g or t
  misc_feature (13)..(14): n is a, c, g or t
  misc_feature (17)..(18): n is a, c, g or t
  misc_feature (22)..(22): n is a, c, g or t
ncatnntanncgnntannatgn

SEQ ID NO: 2 - DNA, 22 bp, artificial sequence
  recognition sequence for E.coli phage N15 and Klebsiella phage Phi K02 protelomerases
ccattatacgcgcgtataatgg

SEQ ID NO: 3 - DNA, 22 bp, artificial sequence
  recognition sequnence for Yersinia phage PY54 protelomerase
gcatactacgcgcgtagtatgc

SEQ ID NO: 4 - DNA, 22 bp, artificial sequence
  recognition sequence for Halomonas phage phiHAP-1 protelomerase
ccatactatacgtatagtatgg

SEQ ID NO: 5 - DNA, 22 bp, artificial sequence
  recognition sequence for Vibrio phage VP882 protelomerase
gcatactatacgtatagtatgc

SEQ ID NO: 6 - DNA, 14 bp, artificial sequence
  recognition sequence for Borrelia burgdorferi protelomerase
attatatatataat

SEQ ID NO: 7 - DNA, 24 bp, artificial sequence
  recognition sequence for Vibrio phage VP882 protelomerase
ggcatactatacgtatagtatgcc

SEQ ID NO: 8 - DNA, 42 bp, artificial sequence
  recognition sequence for Yersinia phage PY54 protelomerase
acctatttcagcatactacgcgcgtagtatgctgaaataggt

SEQ ID NO: 9 - DNA, 90 bp, artificial sequence
  recognition sequence for Halomonas phage phiHAP-1 protelomerase
cctatattgggccacctatgtatgcacagttcgcccatactatacgtatagtatgggcgaactgtgcatacataggtggcccaatatagg

SEQ ID NO: 10 - DNA, 56 bp, Artificial sequence
  TeIN protelomerase recognition sequence
tatcagcacacaattgcccattatacgcgcgtataatggactattgtgtgctgata

SEQ ID NO: 11 - protein, 631 aa, artificial sequence
  NI 5 protelomerase
MSKVKIGELINTLVNEVEAIDASDRPQGDKTKRIKAAAARYKNALFNDKRKFRGKGLQKRITANTFNAYMSRARKRFDDKLHHSFDKNINKLSEKYPLYSEELSSWLSMPTANIRQHMSSLQSKLKEIMPLAEELSNVRIGSKGSDAKIARLIKKYPDWSFALSDLNSDDWKERRDYLYKLFQQGSALLEELHQLKVNHEVLYHLQLSPAERTSIQQRWADVLREKKRNVVVIDYPTYMQSIYDILNNPATLFSLNTRSGMAPLAFALAAVSGRRMIEIMFQGEFAVSGKYTVNFSGQAKKRSEDKSVTRTIYTLCEAKLFVELLTELRSCSAASDFDEVVKGYGKDDTRSENGRINAILAKAFNPWVKSFFGDDRRVYKDSRAIYARIAYEMFFRVDPRWKNVDEDVFFMEILGHDDENTQLHYKQFKLANFSRTWRPEVGDENTRLVALQKLDDEMPGFARGDAGVRLHETVKQLVEQDPSAKITNSTLRAFKFSPTMISRYLEFAADALGQFVGENGQWQLKIETPAIVLPDEESVETIDEPDDESQDDELDEDEIELDEGGGDEPTEEEGPEEHQPTALKPVFKPAKNNGDGTYKIEFEYDGKHYAWSGPADSPMAAMRSAWETYYS

SEQ ID NO: 12 - protein, 1368 aa, artificial sequence [sequence omitted]

SEQ ID NO: 13 - DNA, 4104 bp, artificial sequence
  sequence encoding Cas9
atggataaaaaatatagcattggtctggatattggtaccaatagcgttggttgggcagttattaccgatgaatataaagttccgagcaaaaaatttaaagttctgggtaataccgatcgtcatagcattaaaaaaaatctgattggtgcactgctgtttgatagcggtgaaaccgcagaagcaacccgtctgaaacgtaccgcacgtcgtcgttatacccgtcgtaaaaatcgtatttgttatctgcaggaaatttttagcaatgaaatggcaaaagttgatgatagcttttttcatcgtctggaagaaagctttctggttgaagaagataaaaaacatgaacgtcatccgatttttggtaatattgttgatgaagttgcatatcatgaaaaatatccgaccatttatcatctgcgtaaaaaactggttgatagcaccgataaagcagatctgcgtctgatttatctggcactggcacatatgattaaatttcgtggtcattttctgattgaaggtgatctgaatccggataatagcgatgttgataaactgtttattcagctggttcagacctataatcagctgtttgaagaaaatccgattaatgcaagcggtgttgatgcaaaagcaattctgagcgcacgtctgagcaaaagccgtcgtctggaaaatctgattgcacagctgccgggtgaaaaaaaaaatggtctgtttggtaatctgattgcactgagcctgggtctgaccccgaattttaaaagcaattttgatctggcagaagatgcaaaactgcagctgagcaaagatacctatgatgatgatctggataatctgctggcacagattggtgatcagtatgcagatctgtttctggcagcaaaaaatctgagcgatgcaattctgctgagcgatattctgcgtgttaataccgaaattaccaaagcaccgctgagcgcaagcatgattaaacgttatgatgaacatcatcaggatctgaccctgctgaaagcactggttcgtcagcagctgccggaaaaatataaagaaattttttttgatcagagcaaaaatggttatgcaggttatattgatggtggtgcaagccaggaagaattttataaatttattaaaccgattctggaaaaaatggatggtaccgaagaactgctggttaaactgaatcgtgaagatctgctgcgtaaacagcgtacctttgataatggtagcattccgcatcagattcatctgggtgaactgcatgcaattctgcgtcgtcaggaagatttttatccgtttctgaaagataatcgtgaaaaaattgaaaaaattctgacctttcgtattccgtattatgttggtccgctggcacgtggtaatagccgttttgcatggatgacccgtaaaagcgaagaaaccattaccccgtggaattttgaagaagttgttgataaaggtgcaagcgcacagagctttattgaacgtatgaccaattttgataaaaatctgccgaatgaaaaagttctgccgaaacatagcctgctgtatgaatattttaccgtttataatgaactgaccaaagttaaatatgttaccgaaggtatgcgtaaaccggcatttctgagcggtgaacagaaaaaagcaattgttgatctgctgtttaaaaccaatcgtaaagttaccgttaaacagctgaaagaagattattttaaaaaaattgaatgttttgatagcgttgaaattagcggtgttgaagatcgttttaatgcaagcctgggtacctatcatgatctgctgaaaattattaaagataaagattttctggataatgaagaaaatgaagatattctggaagatattgttctgaccctgaccctgtttgaagatcgtgaaatgattgaagaacgtctgaaaacctatgcacatctgtttgatgataaagttatgaaacagctgaaacgtcgtcgttataccggttggggtcgtctgagccgtaaactgattaatggtattcgtgataaacagagcggtaaaaccattctggattttctgaaaagcgatggttttgcaaatcgtaattttatgcagctgattcatgatgatagcctgacctttaaagaagatattcagaaagcacaggttagcggtcagggtgatagcctgcatgaacatattgcaaatctggcaggtagcccggcaattaaaaaaggtattctgcagaccgttaaagttgttgatgaactggttaaagttatgggtcgtcataaaccggaaaatattgttattgaaatggcacgtgaaaatcagaccacccagaaaggtcagaaaaatagccgtgaacgtatgaaacgtattgaagaaggtattaaagaactgggtagccagattctgaaagaacatccggttgaaaatacccagctgcagaatgaaaaactgtatctgtattatctgcagaatggtcgtgatatgtatgttgatcaggaactggatattaatcgtctgagcgattatgatgttgatcatattgttccgcagagctttctgaaagatgatagcattgataataaagttctgacccgtagcgataaaaatcgtggtaaaagcgataatgttccgagcgaagaagttgttaaaaaaatgaaaaattattggcgtcagctgctgaatgcaaaactgattacccagcgtaaatttgataatctgaccaaagcagaacgtggtggtctgagcgaactggataaagcaggttttattaaacgtcagctggttgaaacccgtcagattaccaaacatgttgcacagattctggatagccgtatgaataccaaatatgatgaaaatgataaactgattcgtgaagttaaagttattaccctgaaaagcaaactggttagcgattttcgtaaagattttcagttttataaagttcgtgaaattaataattatcatcatgcacatgatgcatatctgaatgcagttgttggtaccgcactgattaaaaaatatccgaaactggaaagcgaatttgtttatggtgattataaagtttatgatgttcgtaaaatgattgcaaaaagcgaacaggaaattggtaaagcaaccgcaaaatattttttttatagcaatattatgaatttttttaaaaccgaaattaccctggcaaatggtgaaattcgtaaacgtccgctgattgaaaccaatggtgaaaccggtgaaattgtttgggataaaggtcgtgattttgcaaccgttcgtaaagttctgagcatgccgcaggttaatattgttaaaaaaaccgaagttcagaccggtggttttagcaaagaaagcattctgccgaaacgtaatagcgataaactgattgcacgtaaaaaagattgggatccgaaaaaatatggtggttttgatagcccgaccgttgcatatagcgttctggttgttgcaaaagttgaaaaaggtaaaagcaaaaaactgaaaagcgttaaagaactgctgggtattaccattatggaacgtagcagctttgaaaaaaatccgattgattttctggaagcaaaaggttataaagaagttaaaaaagatctgattattaaactgccgaaatatagcctgtttgaactggaaaatggtcgtaaacgtatgctggcaagcgcaggtgaactgcagaaaggtaatgaactggcactgccgagcaaatatgttaattttctgtatctggcaagccattatgaaaaactgaaaggtagcccggaagataatgaacagaaacagctgtttgttgaacagcataaacattatctggatgaaattattgaacagattagcgaatttagcaaacgtgttattctggcagatgcaaatctggataaagttctgagcgcatataataaacatcgtgataaaccgattcgtgaacaggcagaaaatattattcatctgtttaccctgaccaatctgggtgcaccggcagcatttaaatattttgataccaccattgatcgtaaacgttataccagcaccaaagaagttctggatgcaaccctgattcatcagagcattaccggtctgtatgaaacccgtattgatctgagccagctgggtggtgat

SEQ ID NO: 14 - protein, 1082 aa, Geobacillus thermodenitrificans T12
MKYKIGLDIGITSIGWAVINLDIPRIEDLGVRIFDRAENPKTGESLALPRRLARSARRRLRRRKHRLERIRRLFVREGILTKEELNKLFEKKHEIDVWQLRVEALDRKLNNDELARILLHLAKRRGFRSNRKSERTNKENSTMLKHIEENQSILSSYRTVAEMVVKDPKFSLHKRNKEDNYTNTVARDDLEREIKLIFAKQREYGNIVCTEAFEHEYISIWASQRPFASKDDIEKKVGFCTFEPKEKRAPKATYTFQSFTVWEHINKLRLVSPGGIRALTDDERRLIYKQAFHKNKITFHDVRTLLNLPDDTRFKGLLYDRNTTLKENEKVRFLELGAYHKIRKAIDSVYGKGAAKSFRPIDFDTFGYALTMFKDDTDIRSYLRNEYEQNGKRMENLADKVYDEELIEELLNLSFSKFGHLSLKALRNILPYMEQGEVYSTACERAGYTFTGPKKKQKTVLLPNIPPIANPVVMRALTQARKVVNAIIKKYGSPVSIHIELARELSQSFDERRKMQKEQEGNRKKNETAIRQLVEYGLTLNPTGLDIVKFKLWSEQNGKCAYSLQPIEIERLLEPGYTEVDHVIPYSRSLDDSYTNKVLVLTKENREKGNRTPAEYLGLGSERWQQFETFVLTNKQFSKKKRDRLLRLHYDENEENEFKNRNLNDTRYISRFLANFIREHLKFADSDDKQKVYTVNGRITAHLRSRWNFNKNREESNLHHAVDAAIVACTTPSDIARVTAFYQRREQNKELSKKTDPQFPQPWPHFADELQARLSKNPKESIKALNLGNYDNEKLESLQPVFVSRMPKRSITGAAHQETLRRYIGIDERSGKIQTVVKKKLSEIQLDKTGHFPMYGKESDPRTYEAIRQRLLEHNNDPKKAFQEPLYKPKKNGELGPIIRTIKIIDTTNQVIPLNDGKTVAYNSNIVRVDVFEKDGKYYCVPIYTIDMMKGILPNKAIEPNKPYSEWKEMTEDYTFRFSLYPNDLIRIEFPREKTIKTAVGEEIKIKDLFAYYQTIDSSNGGLSLVSHDNNFSLRSIGSRTLKRFEKYQVDVLGNIYKVRGEKRVGVASSSHSKAGETIRPL

SEQ ID NO: 15 - protein, 1300 aa, artificial sequence
  FnCpfI
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 16 - DNA, 3900 bp, artificial sequence
  sequence encoding FnCpfI
atgagcatttatcaggaatttgttaataaatatagcctgagcaaaaccctgcgttttgaactgattccgcagggtaaaaccctggaaaatattaaagcacgtggtctgattctggatgatgaaaaacgtgcaaaagattataaaaaagcaaaacagattattgataaatatcatcagttttttattgaagaaattctgagcagcgtttgtattagcgaagatctgctgcagaattatagcgatgtttattttaaactgaaaaaaagcgatgatgataatctgcagaaagattttaaaagcgcaaaagataccattaaaaaacagattagcgaatatattaaagatagcgaaaaatttaaaaatctgtttaatcagaatctgattgatgcaaaaaaaggtcaggaaagcgatctgattctgtggctgaaacagagcaaagataatggtattgaactgtttaaagcaaatagcgatattaccgatattgatgaagcactggaaattattaaaagctttaaaggttggaccacctattttaaaggttttcatgaaaatcgtaaaaatgtttatagcagcaatgatattccgaccagcattatttatcgtattgttgatgataatctgccgaaatttctggaaaataaagcaaaatatgaaagcctgaaagataaagcaccggaagcaattaattatgaacagattaaaaaagatctggcagaagaactgacctttgatattgattataaaaccagcgaagttaatcagcgtgtttttagcctggatgaagtttttgaaattgcaaattttaataattatctgaatcagagcggtattaccaaatttaataccattattggtggtaaatttgttaatggtgaaaataccaaacgtaaaggtattaatgaatatattaatctgtatagccagcagattaatgataaaaccctgaaaaaatataaaatgagcgttctgtttaaacagattctgagcgataccgaaagcaaaagctttgttattgataaactggaagatgatagcgatgttgttaccaccatgcagagcttttatgaacagattgcagcatttaaaaccgttgaagaaaaaagcattaaagaaaccctgagcctgctgtttgatgatctgaaagcacagaaactggatctgagcaaaatttattttaaaaatgataaaagcctgaccgatctgagccagcaggtttttgatgattatagcgttattggtaccgcagttctggaatatattacccagcagattgcaccgaaaaatctggataatccgagcaaaaaagaacaggaactgattgcaaaaaaaaccgaaaaagcaaaatatctgagcctggaaaccattaaactggcactggaagaatttaataaacatcgtgatattgataaacagtgtcgttttgaagaaattctggcaaattttgcagcaattccgatgatttttgatgaaattgcacagaataaagataatctggcacagattagcattaaatatcagaatcagggtaaaaaagatctgctgcaggcaagcgcagaagatgatgttaaagcaattaaagatctgctggatcagaccaataatctgctgcataaactgaaaatttttcatattagccagagcgaagataaagcaaatattctggataaagatgaacatttttatctggtttttgaagaatgttattttgaactggcaaatattgttccgctgtataataaaattcgtaattatattacccagaaaccgtatagcgatgaaaaatttaaactgaattttgaaaatagcaccctggcaaatggttgggataaaaataaagaaccggataataccgcaattctgtttattaaagatgataaatattatctgggtgttatgaataaaaaaaataataaaatttttgatgataaagcaattaaagaaaataaaggtgaaggttataaaaaaattgtttataaactgctgccgggtgcaaataaaatgctgccgaaagttttttttagcgcaaaaagcattaaattttataatccgagcgaagatattctgcgtattcgtaatcatagcacccataccaaaaatggtagcccgcagaaaggttatgaaaaatttgaatttaatattgaagattgtcgtaaatttattgatttttataaacagagcattagcaaacatccggaatggaaagattttggttttcgttttagcgatacccagcgttataatagcattgatgaattttatcgtgaagttgaaaatcagggttataaactgacctttgaaaatattagcgaaagctatattgatagcgttgttaatcagggtaaactgtatctgtttcagatttataataaagattttagcgcatatagcaaaggtcgtccgaatctgcataccctgtattggaaagcactgtttgatgaacgtaatctgcaggatgttgtttataaactgaatggtgaagcagaactgttttatcgtaaacagagcattccgaaaaaaattacccatccggcaaaagaagcaattgcaaataaaaataaagataatccgaaaaaagaaagcgtttttgaatatgatctgattaaagataaacgttttaccgaagataaatttttttttcattgtccgattaccattaattttaaaagcagcggtgcaaataaatttaatgatgaaattaatctgctgctgaaagaaaaagcaaatgatgttcatattctgagcattgatcgtggtgaacgtcatctggcatattataccctggttgatggtaaaggtaatattattaaacaggatacctttaatattattggtaatgatcgtatgaaaaccaattatcatgataaactggcagcaattgaaaaagatcgtgatagcgcacgtaaagattggaaaaaaattaataatattaaagaaatgaaagaaggttatctgagccaggttgttcatgaaattgcaaaactggttattgaatataatgcaattgttgtttttgaagatctgaattttggttttaaacgtggtcgttttaaagttgaaaaacaggtttatcagaaactggaaaaaatgctgattgaaaaactgaattatctggtttttaaagataatgaatttgataaaaccggtggtgttctgcgtgcatatcagctgaccgcaccgtttgaaacctttaaaaaaatgggtaaacagaccggtattatttattatgttccggcaggttttaccagcaaaatttgtccggttaccggttttgttaatcagctgtatccgaaatatgaaagcgttagcaaaagccaggaattttttagcaaatttgataaaatttgttataatctggataaaggttattttgaatttagctttgattataaaaattttggtgataaagcagcaaaaggtaaatggaccattgcaagctttggtagccgtctgattaattttcgtaatagcgataaaaatcataattgggatacccgtgaagtttatccgaccaaagaactggaaaaactgctgaaagattatagcattgaatatggtcatggtgaatgtattaaagcagcaatttgtggtgaaagcgataaaaaattttttgcaaaactgaccagcgttctgaataccattctgcagatgcgtaatagcaaaaccggtaccgaactggattatctgattagcccggttgcagatgttaatggtaatttttttgatagccgtcaggcaccgaaaaatatgccgcaggatgcagatgcaaatggtgcatatcatattggtctgaaaggtctgatgctgctgggtcgtattaaaaataatcaggaaggtaaaaaactgaatctggttattaaaaatgaagaatattttgaatttgttcagaatcgtaataat

SEQ ID NO: 17 - protein, 1263 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 18 - protein, 1274 aa, artificial sequence
  MAD7-NLS
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLSGGSPKKKRKV

SEQ ID NO: 19 - DNA, 115 bp, artificial sequence
  oligonucleotide
aggaccggatcaacttatcagcacacaattgcccattatacgcgcgtataatggactattgtgtgctgataaagaaagttgtcggtgtctttgtgagatgtgtataagagacagt

SEQ ID NO: 20 - DNA, 114 bp, artificial sequence
  oligonucleotide
ctgtctcttatacacatctcacaaagacaccgacaactttctttatcagcacacaatagtccattatacgcgcgtataatgggcaattgtgtgctgataagttgatccggtcct

SEQ ID NO: 21 - DNA, 21 bp, artificial sequence
  forward primer
tcacgctgatttacagcggca

SEQ ID NO: 22 - DNA, 20 bp, artificial sequence
  reverse primer
cgatgctgattgccgttccg

(19) United States
(12) Patent Application Publication
(10) Pub. No.: US 2022/0333100 A1
(43) Pub. Date: Oct. 20, 2022

(54) NGS LIBRARY PREPARATION USING COVALENTLY CLOSED NUCLEIC ACID MOLECULE ENDS

(71) Applicant: Keygene N.V., Wageningen (NL)

(72) Inventors: René Cornelis Josephus HOGERS, Wageningen (NL); Stefan John WHITE, Wageningen (NL)

(21) Appl. No.: 17/843,612

(22) Filed: Jun. 17, 2022

Related U.S. Application Data
(63) Continuation of application No. PCT/EP2020/086887, filed on Dec. 17, 2020.

(30) Foreign Application Priority Data
Dec. 20, 2019 (EP) 19218832.4

(51) Int. Cl. C12N 15/10; C12Q 1/6806; C12Q 1/6855

(52) U.S. Cl. CPC C12N 15/1093; C12Q 1/6806; C12Q 1/6855

(57) ABSTRACT

The current invention pertains to adapters comprising a protelomerase recognition sequence, preferably a TeIN protelomerase recognition sequence. The adapters of the invention can be used for the preparation of a nucleic acid molecule library. The invention also relates to a method for producing a nucleic acid molecule library using one or more adapters comprising a protelomerase recognition sequence. The adapters may be contacted with a protelomerase to cleave and close the ends of the adapters. Said closed adapters are e.g. protected against exonuclease treatment. The method of the invention further concerns an amplification method and a sequencing method using adapters having a protelomerase recognition sequence.

### CROSS-REFERENCE TO RELATED APPLICATIONS

The present application is a Continuation of International Patent Application No. PCT/EP2020/086887, filed Dec. 17, 2020, which claims priority to: Europe Patent Application No. 19218832.4 filed Dec. 20, 2019, the entire contents of both of which are hereby incorporated by reference.

### SEQUENCE LISTING

The instant application contains a Sequence Listing which has been submitted electronically in ASCII format and is hereby incorporated by reference in its entirety. Said ASCII copy, created on Jun. 16, 2022, is named 085342-4200_SequenceListing.txt and is 74,653 bytes in size.

### FIELD OF THE INVENTION

The present invention is in the field of genetic research, more particular in the field of targeted nucleic acid isolation, e.g. for sequence analysis and processing of nucleic acid samples. Disclosed are new methods and means for library preparation and complexity reduction of nucleic acid samples.

### BACKGROUND OF THE INVENTION

A significant component of genetic research is sequence analysis of defined DNA loci, e.g. to genotype known variants, or identify sequence changes or variants. Such analysis often needs to be done in a multiplex fashion, e.g., a specific set of loci needs to be analyzed in a large number of samples.

The ideal assay is flexible with regards to the number of samples and loci that need to be screened, is highly accurate, and is amenable to different sequencing platforms. When analysing a subset of nucleic acids from a collection of fragments, there is often a need for enrichment of the fragments of interest. Enrichment can be performed through selection (e.g. purification or amplification) of the targeted nucleic acids or by removal of unwanted nucleic acids. Ideally enrichment steps are amplification free. For instance, US2014/0134610 describes a complexity reduction method using type II restriction enzymes to fragment nucleic acids in a sample, followed by ligation of protective adapters and subsequently degrading all noncaptured nucleic acid using exonucleases. In WO2016/028887, this method is amended by using a programmable endonuclease, i.e. a CRISPR-endonuclease for fragmenting the nucleic acid in the sample.

In most applications, the first step in next-generation sequencing (NGS) is the preparation of a library. Library preparation for NGS can be performed using various protocols. For long read sequencing libraries using the PacBio platform, hairpin adapters are ligated to the ends of nucleic acid molecules. These hairpin adapters are added to remove all non-adapter ligated molecules using an exonuclease treatment, and to be able to generate sequencing reads that span multiple passes of the input nucleic acid molecules. The latter enables creation of a highly accurate consensus sequence of the sequenced nucleic acid molecule. Addition of the hairpin adapter involves multiple steps, which starts with an optional fragmentation of the input nucleic acid molecules, followed by polishing of the fragment ends and the addition of a 3′-A staggered (or “sticky”) end. Optionally during the polishing step, a repair step can be performed to remove damaged positions (e.g. nicks) in the nucleic acid molecules.

As an alternative to these separate steps, the fragmentation step and adapter addition step can be combined in a single step using a transposase enzyme (“tagmentation”). Tagmentation is widely used in e.g. Illumina Nextera and the Oxford Nanopore Technologies (ONT) rapid library preparation protocols. When performing a repair step after the transposase reaction most nucleic acid fragments contain adapters at their ends. Fragmentation or tagmentation however create fairly random nucleic acid fragments.

It is an objective of the invention to provide a novel method for preparing a nucleic acid molecule library, e.g. for subsequent sequencing and/or cloning, wherein preferably the method comprises a step of enriching the library for a nucleic acid of interest.

### SUMMARY

The invention may be summarized in the following numbered embodiments:

Embodiment 1. An adapter, wherein the adapter is at least partly double-stranded and comprises a protelomerase recognition sequence, preferably a TeIN protelomerase recognition sequence.

Embodiment 2. An adapter according to embodiment 1, wherein the adapter further comprises an identifier sequence.

Embodiment 3. An adapter according to embodiment 1 or 2, wherein the adapter comprises at least one staggered end.

Embodiment 4. A method for preparing a nucleic acid molecule library, wherein the method comprises the steps of:
a) providing a sample comprising at least a first and a second nucleic acid molecule, wherein the first nucleic acid molecule comprises a first target sequence not present in the second nucleic acid molecule and wherein optionally the second nucleic acid molecule comprises a second target sequence;
b) ligating an adapter as defined in any one of embodiments 1-3 to the ends of the first and second nucleic acid molecule to provide adapter ligated nucleic acid molecules;
c) contacting the adapter ligated nucleic acid molecules with a protelomerase, preferably a TeIN protelomerase, to cleave and covalently close the cleaved ends, resulting in a first and second nucleic acid molecule comprising closed ends; and
d) cleaving the first nucleic acid molecule comprising the closed ends at the first target sequence, to provide a first nucleic acid comprising one open end and one closed end.

Embodiment 5. A method according to embodiment 4, wherein the sample in step a) comprises the first and second nucleic acid molecule and a plurality of further nucleic acid molecules.

Embodiment 6. A method according to embodiment 4 or 5, wherein the first nucleic acid molecule in step d) is cleaved by a programmable nuclease or a restriction endonuclease.

Embodiment 7. A method according to embodiment 6, wherein the programmable nuclease is an RNA-guided CRISPR nuclease.

Embodiment 8. A method according to any one of embodiments 4-7, wherein the first and second nucleic acid molecules in step a) are provided by fragmentation, preferably fragmentation of a genomic nucleic acid molecule.

Embodiment 9. A method according to embodiment 8, wherein the adapter in step b) is ligated by tagmentation.

Embodiment 10. A method according to any one of embodiments 4-9, wherein the method comprises a step c1) of exposing the sample to an exonuclease after obtaining the nucleic acid molecules comprising closed ends in step c) and prior to cleaving the first nucleic acid molecule comprising the closed ends in step d).

Embodiment 11. A method according to any one of embodiments 4-9, wherein the method comprises a step e) of exposing the sample to an exonuclease after obtaining the first nucleic acid molecule comprising one open end and one closed end in step d).

Embodiment 12. A method according to embodiment 11, wherein the method comprises a step f) of cleaving the second nucleic acid molecule comprising the closed ends at the second target sequence, resulting in a second nucleic acid comprising one open end and one closed end.

Embodiment 13. A method according to any one of embodiments 4-12, wherein said method comprises a step g) of linking a further adapter to the open end of the first, or optionally second, nucleic acid molecule comprising one open and one closed end, wherein said further adapter comprises at least one of an amplification primer binding site and sequence primer binding site and optionally an identifier sequence.

Embodiment 14. A method according to any one of embodiments 4-13, wherein a nucleic acid molecule library is prepared from a plurality of samples, and wherein preferably the plurality of samples are pooled, preferably prior to step c), step d) , step e), step f) or prior to step g).

Embodiment 15. A method according to embodiment 13, wherein the samples are pooled after step g).

Embodiment 16. A method according to any one of embodiments 4-13, wherein in step b) the adapter ligated nucleic acid molecules are repaired to remove single-stranded breaks prior to contacting the molecules with a TeIN protelomerase in step c).

Embodiment 17. A method for amplification of a nucleic acid molecule library, wherein the method comprises the steps of
preparing nucleic acid molecule library as defined in any one of embodiments 13-16; and
amplifying the nucleic acid molecule library using at least one of:
   i) a first, and optionally a second, primer annealing to the first nucleic acid molecule comprising one open and one closed end as obtained in step d);
   ii) a first, and optionally a second, primer annealing to the second nucleic acid molecule comprising one open and one closed end as obtained in step f);
   iii) a first, and optionally a second, primer annealing to the further adapter as defined in step g); and
   iv) a combination of a first primer as defined in i) or ii), and a second primer as defined in iii).

Embodiment 18. A method for analysing a sequence of interest in a sample comprising a first and a second nucleic acid molecule, comprising the steps of:
preparing a nucleic acid molecule library as defined in any one of embodiments 13-16;
optionally amplifying the prepared nucleic acid molecule as defined in embodiment 17; and
sequencing, preferably deep-sequencing, the nucleic acid molecule library.

Embodiment 19. A kit of parts comprising:
one or more adapters as defined in any one of embodiments 1-3; and
optionally a protelomerase, preferably a TeIN protelomerase.

### Definitions

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

Methods of carrying out the conventional techniques used in methods of the invention will be evident to the skilled worker. The practice of conventional techniques in molecular biology, biochemistry, computational chemistry, cell culture, recombinant DNA, bioinformatics, genomics, sequencing and related fields are well-known to those of skill in the art and are discussed, for example, in the following literature references: Sambrook et al. Molecular Cloning. A Laboratory Manual, 2nd Edition, Cold Spring Harbor Laboratory Press, Cold Spring Harbor, N. Y., 1989; Ausubel et al. Current Protocols in Molecular Biology, John Wiley & Sons, New York, 1987 and periodic updates; and the series Methods in Enzymology, Academic Press, San Diego.

“A,” “an,” and “the”: these singular form terms include plural referents unless the content clearly dictates otherwise. Thus, for example, reference to “a cell” includes a combination of two or more cells, and the like.

As used herein, the term “about” is used to describe and account for small variations. For example, the term can refer to less than or equal to ±10%, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

As used herein, the term “adapter” is a single-stranded, double-stranded, partly double-stranded, Y-shaped or hairpin nucleic acid molecule that can be attached, preferably ligated, to the end of other nucleic acids, e.g., to one or both strands of a double-stranded DNA molecule, and preferably has a limited length, e.g., about 10 to about 200, or about 10 to about 100 bases, or about 10 to about 80, or about 10 to about 50, or about 10 to about 30 base pairs in length, and is preferably chemically synthesized. The double-stranded structure of the adapter may be formed by two distinct oligonucleotide molecules that are base paired with one another, or by a hairpin structure of a single oligonucleotide strand. As would be apparent, the attachable end of an adapter may be designed to be compatible with, and optionally ligatable to, overhangs made by cleavage by a restriction enzyme and/or programmable nuclease, may be designed to be compatible with an overhang created after addition of a non-template elongation reaction (e.g., 3′-A addition), or may have blunt ends.

“And/or”: the term “and/or” refers to a situation wherein one or more of the stated cases may occur, alone or in combination with at least one of the stated cases, up to with all of the stated cases.

“Amplification” used in reference to a nucleic acid or nucleic acid reactions, refers to in vitro methods of making copies of a particular nucleic acid, such as a target nucleic acid, or a tagged nucleic acid. Numerous methods of amplifying nucleic acids are known in the art, and amplification reactions include polymerase chain reactions, ligase chain reactions, strand displacement amplification reactions, rolling circle amplification reactions, transcription-mediated amplification methods such as NASBA (e.g., U.S. Pat. No. 5,409,818), loop mediated amplification methods (e.g., “LAMP” amplification using loop-forming sequences, e.g., as described in U.S. Pat. No. 6,410,278) and isothermal amplification reactions. The nucleic acid that is amplified can be DNA comprising, consisting of, or derived from DNA or RNA or a mixture of DNA and RNA, including modified DNA and/or RNA. The products resulting from amplification of a nucleic acid molecule or molecules (i.e., “amplification products”), whether the starting nucleic acid is DNA, RNA or both, can be either DNA or RNA, or a mixture of both DNA and RNA nucleosides or nucleotides, or they can comprise modified DNA or RNA nucleosides or nucleotides.

A “copy” can be, but is not limited to, a sequence having full sequence complementarity or full sequence identity to a particular sequence. Alternatively, a copy does not necessarily have perfect sequence complementarity or identity to this particular sequence, e.g. a certain degree of sequence variation is allowed. For example, copies can include nucleotide analogs such as deoxyinosine or deoxyuridine, intentional sequence alterations (such as sequence alterations introduced through a primer comprising a sequence that is hybridizable, but not complementary, to a particular sequence), and/or sequence errors that occur during amplification.

The term “complementarity” is herein defined as the sequence identity of a sequence to a fully complementary strand (e.g. the second, or reverse, strand). For example, a sequence that is 100% complementary (or fully complementary) is herein understood as having 100% sequence identity with the complementary strand and e.g. a sequence that is 80% complementary is herein understood as having 80% sequence identity to the (fully) complementary strand.

“Comprising”: this term is construed as being inclusive and open ended, and not exclusive. Specifically, the term and variations thereof mean the specified features, steps or components are included. These terms are not to be interpreted to exclude the presence of other features, steps or components.

“Construct” or “nucleic acid construct” or “vector”: this refers to a man-made nucleic acid molecule resulting from the use of recombinant DNA technology and which can be used to deliver exogenous DNA into a host cell, often with the purpose of expression in the host cell of a DNA region comprised on the construct. The vector backbone of a construct may for example be a plasmid into which a (chimeric) gene is integrated or, if a suitable transcription regulatory sequence is already present (for example a (inducible) promoter), only a desired nucleotide sequence (e.g., a coding sequence) is integrated downstream of the transcription regulatory sequence. Vectors may comprise further genetic elements to facilitate their use in molecular cloning, such as e.g., selectable markers, multiple cloning sites and the like.

The terms “double-stranded” and “duplex” as used herein, describes two complementary polynucleotides that are base-paired, i.e., hybridized together. Complementary nucleotide strands are also known in the art as reverse-complement.

The term “effective amount,” as used herein, refers to an amount of a biologically active agent that is sufficient to elicit a desired biological effect. For example, in some embodiments, an effective amount of an exonuclease may refer to the amount of the exonuclease that is sufficient to induce cleavage of an unprotected nucleic acid. As will be appreciated by the skilled artisan, the effective amount of an agent may vary depending on various factors such as the agent being used, the conditions wherein the agent is used, and the desired biological effect, e.g. degree of nuclease cleavage to be detected.

“Exemplary”: this terms means “serving as an example, instance, or illustration,” and should not be construed as excluding other configurations disclosed herein.

“Expression”: this refers to the process wherein a DNA region, which is operably linked to appropriate regulatory regions, particularly a promoter, is transcribed into an RNA, which in turn can be translated into a protein or peptide.

A “guide sequence” is to be understood herein as a sequence that directs an RNA or DNA guided endonuclease to a specific site in an RNA or DNA molecule. In the context of a gRNA-CAS complex, “guide sequence” is further to be understood herein as the section of the sgRNA or crRNA, which is required for targeting a gRNA-CAS complex to a specific site in a duplex DNA.

A gRNA-CAS complex is to be understood herein a CAS protein, also named a CRISPR-endonuclease or CRISPR-nuclease, which is complexed or hybridized to a guide RNA, wherein the guide RNA may be a crRNA and/or a tracrRNA, or a sgRNA.

“Identity” and “similarity” can be readily calculated by known methods. “Sequence identity” and “sequence similarity” can be determined by alignment of two peptide or two nucleotide sequences using global or local alignment algorithms, depending on the length of the two sequences. Sequences of similar lengths are preferably aligned using a global alignment algorithm (e.g. Needleman Wunsch) which aligns the sequences optimally over the entire length, while sequences of substantially different lengths are preferably aligned using a local alignment algorithm (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blosum62 for proteins and DNAFull for DNA). When sequences have a substantially different overall lengths, local alignments, such as those using the Smith Waterman algorithm, are preferred.

Alternatively, percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

The term “nucleotide” includes, but is not limited to, naturally-occurring nucleotides, including guanine, cytosine, adenine and thymine (G, C, A and T, respectively). The term “nucleotide” is further intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The terms “nucleic acid”, “polynucleotide” and “nucleic acid molecule” are used interchangeably herein to describe a polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides or ribonucleotides, and may be produced enzymatically or synthetically (e.g., PNA as described in U.S. Pat. No. 5,948,902 and the references cited therein). The nucleic acid may hybridize with naturally occurring nucleic acids in a sequence specific manner analogous to that of two naturally occurring nucleic acids, e.g., can participate in Watson-Crick base pairing interactions. In addition, nucleic acids and polynucleotides may be isolated (and optionally subsequently fragmented) from cells, tissues and/or bodily fluids. The nucleic acid can be e.g. genomic DNA (gDNA), mitochondrial, cell free DNA (cfDNA), DNA from a library and/or RNA from a library.

The term “nucleic acid sample” or “sample comprising a nucleic acid” as used herein denotes any sample containing a nucleic acid, wherein a sample relates to a material or mixture of materials, typically, although not necessarily, in liquid form, containing one or more nucleic acid molecules of interest. The one or more nucleic acid molecules of interest preferably comprise a sequence of interest. The nucleic acid molecule of interest is preferably the first nucleic acid molecule or the second nucleic acid molecule a defined herein. The nucleic acid sample preferably comprises a sequence of interest. The nucleic acid sample used as starting material in the method of the invention can be from any source, e.g., a whole genome, a collection of chromosomes, a single chromosome, one or more regions from one or more chromosomes or transcribed genes, and may be purified directly from the biological source or from a laboratory source, e.g., a nucleic acid library. The nucleic acid samples can be obtained from the same individual, which can be a human or other species (e.g., plant, bacteria, fungi, algae, archaea, etc.), or from different individuals of the same species, or different individuals of different species. For example, the nucleic acid samples may be from a cell, tissue, biopsy, bodily fluid, genome DNA library, cDNA library and/or a RNA library. The nucleic acid sample preferably comprises at least a first nucleic acid molecule and a second nucleic acid molecule.

The term “sequence of interest”, includes, but is not limited to, any genetic sequence preferably present within a cell, such as, for example a gene, part of a gene, or a non-coding sequence within or adjacent to a gene. The sequence of interest may be present in a chromosome, an episome, an organellar genome such as mitochondrial or chloroplast genome or genetic material that can exist independently to the main body of genetic material such as an infecting viral genome, plasmids, episomes, transposons for example. A sequence of interest may be within the coding sequence of a gene, within transcribed non-coding sequence such as, for example, leader sequences, trailer sequence or introns. Said nucleic acid sequence of interest may be present in a double or a single strand nucleic acid. Preferably, the sequence of interest is present in the first nucleic acid molecule or in the second nucleic acid molecule.

The sequence of interest can be, but is not limited to, a sequence having or suspected of having, a polymorphism, e.g. a SNP.

The term “oligonucleotide” as used herein denotes a single-stranded multimer of nucleotides, preferably of about 2 to 200 nucleotides, or up to 500 nucleotides in length. Oligonucleotides may be synthetic or may be made enzymatically, and, in some embodiments, are about 10 to 50 nucleotides in length. Oligonucleotides may contain ribonucleotide monomers (i.e., may be oligoribonucleotides) or deoxyribonucleotide monomers. An oligonucleotide may be about 10 to 20, 20 to 30, 30 to 40, 40 to 50, 50 to 60, 60 to 70, 70 to 80, 80 to 100, 100 to 150, 150 to 200, or about 200 to 250 nucleotides in length, for example.

“Plant”: this includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, grains and the like. Non-limiting examples of plants include crop plants and cultivated plants, such as barley, cabbage, canola, cassava, cauliflower, chicory, cotton, cucumber, eggplant, grape, hot pepper, lettuce, maize, melon, oilseed rape, potato, pumpkin, rice, rye, sorghum, squash, sugar cane, sugar beet, sunflower, sweet pepper, tomato, water melon, wheat, and zucchini.

The “protospacer sequence” is the sequence that is recognized or hybridizable to a guide sequence within a guide RNA, more specifically the crRNA or, in case of a sgRNA, the crRNA part of the guide RNA. In the context of the invention it is understood herein that the “protospacer sequence” is an example of a target sequence, i.e. a sequence present in the first or second nucleic acid molecule as defined herein.

An “endonuclease” is an enzyme that hydrolyses at least one strand of a duplex DNA or a strand of an RNA molecule, upon binding to its target or recognition site. An endonuclease is to be understood herein as a site-specific endonuclease and the terms “endonuclease” and “nuclease” are used interchangeable herein. A restriction endonuclease is to be understood herein as an endonuclease that hydrolyses both strands of the duplex at the same time to introduce a double strand break in the DNA. A “nicking” endonuclease is an endonuclease that hydrolyses only one strand of the duplex to produce DNA molecules that are “nicked” rather than cleaved.

An “exonuclease” is defined herein as any enzyme that cleaves one or more nucleotides from the end (exo) of a polynucleotide.

“Reducing complexity” or “complexity reduction” is to be understood herein as the reduction of a complex nucleic acid sample, such as samples derived from genomic DNA, cfDNA derived from liquid biopsies, isolated RNA samples and the like. Reduction of complexity results in the enrichment of one or more specific nucleic acids, preferably comprising a sequence of interest, comprised within the complex starting material and/or the generation of a subset of the sample, wherein the subset comprises or consists of one or more specific nucleic acids, preferably comprising a sequence of interest, comprised within the complex starting material, while non-specific nucleic acids, preferably not comprising a sequence of interest, are reduced in amount by at least 20%, 30%, 40%, 50%, 60%, 70%, 80%, 90%, 91%, 92%, 93%, 94%, 95%, 96%, 97%, 98% or 99% as compared to the amount of non-specific nucleic acids in the starting material, i.e. before complexity reduction.

Reduction of complexity is in general performed prior to further analysis or method steps, such as amplification, barcoding, sequencing, determining epigenetic variation etc. Preferably complexity reduction is reproducible complexity reduction, which means that when the same sample is reduced in complexity using the same method, the same, or at least comparable, subset is obtained, as opposed to random complexity reduction.

Examples of complexity reduction methods include for example AFLP® (Keygene N.V., the Netherlands; see e.g., EP 0 534 858), Arbitrarily Primed PCR amplification, capture-probe hybridization, the methods described by Dong (see e.g., WO 03/012118, WO 00/24939) and indexed linking (Unrau P. and Deugau K. V. (1994) Gene 145:163-169), the methods described in WO2006/137733; WO2007/037678; WO2007/073165; WO2007/073171, US 2005/260628, WO 03/010328, US 2004/10153, genome portioning (see e.g. WO 2004/022758), Serial Analysis of Gene Expression (SAGE; see e.g. Velculescu et al., 1995, see above, and Matsumura et al., 1999, The Plant Journal, vol. 20 (6) : 719-726) and modifications of SAGE (see e.g. Powell, 1998, Nucleic Acids Research, vol. 26 (14): 3445-3446; and Kenzelmann and Mühlemann, 1999, Nucleic Acids Research, vol. 27 (3) : 917-918) , MicroSAGE (see e.g. Datson et al., 1999, Nucleic Acids Research, vol. 27 (5) : 1300-1307), Massively Parallel Signature Sequencing (MPSS; see e.g. Brenner et al., 2000, Nature Biotechnology, vol. 18:630-634 and Brenner et al., 2000, PNAS, vol. 97 (4) :1665-1670), self-subtracted cDNA libraries (Laveder et al., 2002, Nucleic Acids Research, vol. 30(9):e38), Real-Time Multiplex Ligation-dependent Probe Amplification (RT-MLPA; see e.g. Eldering et al., 2003, vol. 31 (23) : el53) , High Coverage Expression Profiling (HiCEP; see e.g. Fukumura et al. , 2003, Nucleic Acids Research, vol. 31(16) :e94), a universal micro-array system as disclosed in Roth et al.(Roth et al., 2004, Nature Biotechnology, vol. 22 (4): 418-426), a transcriptome subtraction method (see e.g. Li et al., Nucleic Acids Research, vol. 33 (16) : e136), and fragment display (see e.g. Metsis et al., 2004, Nucleic Acids Research, vol. 32 (16) : e127).

“Sequence” or “Nucleotide sequence”: This refers to the order of nucleotides of, or within a nucleic acid. In other words, any order of nucleotides in a nucleic acid may be referred to as a sequence or nucleic acid sequence. For example, the target sequence is an order of nucleotides comprised in a single strand of a DNA duplex.

The term “sequencing,” as used herein, refers to a method by which the identity of at least 10 consecutive nucleotides (e.g., the identity of at least 20, at least 50, at least 100 or at least 200 or more consecutive nucleotides) of a polynucleotide are obtained. The term “next-generation sequencing” refers to the so-called parallelized sequencing-by-synthesis or sequencing-by-ligation platforms, e.g., such as currently employed by Illumina, Life Technologies, PacBio and Roche etc. Next-generation sequencing methods may also include nanopore sequencing methods, such as those commercialized by Oxford Nanopore Technologies (ONT), or electronic-detection based methods such as Ion Torrent technology commercialized by Life Technologies. Preferably, the next-generation sequencing method is a nanopore sequencing method, preferably a nanopore selective sequencing method.

“Nanopore selective sequencing” is to be understood herein as selectively sequencing of single molecules in real time using nanopore sequencing technology such as from Oxford Nanopore or Ontera, and mapping streaming nanopore current signals or base calls to a reference sequence in order to reject non-target sequences. In response to the data being generated, the sequencer is steered to either pursue sequencing of a nucleic acid, or to quit and remove the nucleic acid from the sequencing pore by reversing the polarity of the voltage across the specific pore for a certain short period of time sufficient to eject the non-target molecule and make the nanopore available for a new sequencing read. Examples of Nanopore selective sequencing methods are described in Payne et al., 2020 (Nanopore adaptive sequencing for mixed samples, whole exome capture and targeted panels, Feb. 3, 2020; DOI: 10.1101/2020.02.03.926956) and Kovaka et al. 2020 (Targeted nanopore sequencing by real-time mapping of raw electrical signal with UNCALLED, Feb. 3, 2020; doi: 10.1101/2020.02.03.931923), which are incorporated herein by reference.

A “first nucleic acid molecule” in the context of the invention may be a small or longer stretch, or selected portion of a nucleic acid, single or double stranded. Prior to the performing the method of the invention, the first nucleic acid molecule may be comprised within a larger nucleic acid molecule, e.g. within a larger nucleic acid molecule present in a sample to be analysed. Preferably, the first nucleic acid molecule comprises a first target sequence.

A “second nucleic acid molecule” in the context of the invention may be a small or longer stretch, or selected portion of a nucleic acid, single or double stranded. Prior to the performing the method of the invention, the second nucleic acid molecule may be comprised within a larger nucleic acid molecule, e.g. within a larger nucleic acid molecule present in a sample to be analysed. The first nucleic acid molecule may be present in the same larger nucleic acid molecule. Alternatively, the first and second nucleic acid molecules are present in separate larger nucleic acid molecules, wherein the separate larger nucleic acid molecules are present in the same sample. In some embodiments, the second nucleic acid molecule may comprise a second target sequence.

At least one of the first and second nucleic acid molecule may comprise a sequence of interest. Preferably, the first nucleic acid molecule comprises a sequence of interest. In an alternative embodiment, the second nucleic acid molecule comprises the sequence of interest.

The sequence of interest may be any sequence within a nucleic acid sample, e.g., a gene, gene complex, locus, pseudogene, regulatory region, highly repetitive region, polymorphic region, or portion thereof. The sequence of interest may also be a region comprising genetic or epigenetic variations indicative for a phenotype or disease. A sequence of interest is preferably the object of a further analysis or action, such as, but not limited to copying, amplification, sequencing and/or other procedure for nucleic acid interrogation.

A “target sequence” is defined herein as a sequence present in the first or second nucleic acid molecule as defined herein, which sequence is recognized by at least one of a nuclease and nickase as defined herein.

In some aspects, a plurality or “set” of nucleic acid molecules used in the method of the invention comprise one or more sequences of interest that are selected to be enriched. Optionally, such set consists of structurally or functionally related nucleic acid molecules. A nucleic acid molecule in the context of the invention can comprise both natural and non-natural, artificial, or non-canonical nucleotides including, but not limited to, DNA, RNA, BNA (bridged nucleic acid), LNA (locked nucleic acid), PNA (peptide nucleic acid), morpholino nucleic acid, glycol nucleic acid, threose nucleic acid, epigenetically modified nucleotide such as methylated DNA, and mimetics and combinations thereof.

Preferably, the sequence of interest is a small or longer contiguous stretch of nucleotides (i.e. a polynucleotide) of a single strand of duplex DNA, wherein said duplex DNA further comprises a complementary strand comprising a sequence complementary to the sequence of interest. Preferably, said duplex DNA is genomic DNA (gDNA) and/or cell free DNA (cfDNA).

### DETAILED DESCRIPTION OF THE INVENTION

The inventors discovered that adapters comprising a Protelomerase recognition site can be used for library preparation. In particular, adapters containing a recognition site for the Protelomerase enzyme can be ligated to nucleic acid molecules, wherein these nucleic acid molecules are either double stranded or made double stranded after adapter ligation. These adapters are subsequently cut by the Protelomerase enzyme and simultaneously the ends of the nucleic acid molecules are covalently closed. In case both ends of a nucleic acid molecule are closed this way, the molecule is protected against exonuclease degradation as it lacks free “end” nucleotides.

A terminus of a double stranded nucleic acid, wherein the 3′-end terminal nucleotide of the respective upper strand is covalently linked to the 5′-end terminal nucleotide of the respective bottom strand, is annotated herein as a “closed end”. Likewise, a terminus of a double stranded nucleic acid, wherein the 5′-end terminal nucleotide of the respective upper strand is covalently linked to the 3′-end terminal nucleotide of the respective bottom strand, is also annotated herein as a “closed end”. A “closed end” is thus understood herein as a terminus of a double stranded nucleic acid wherein said terminal nucleic acids from opposite strands are covalently linked to each other, as opposed to an “open end” which is understood herein as a terminus of a double stranded nucleic acid wherein said terminal nucleic acids from opposite strands are not covalently linked to each other.

For the novel library preparation methods detailed herein, preferably all nucleic acid molecules that are present in a particular nucleic acid sample are tagged on both sides with a Protelomerase adapter and are thus cut upon Protelomerase treatment, rendering covalently closed nucleic acid molecules that are insensitive for 5′ or 3′ modifying enzymes. An optional step of exonuclease treatment of the Protelomerase-treated sample can be added to remove any possible nucleic acid molecules that are not covalently closed on both ends. Subsequently, the (covalently closed) nucleic acid molecules can be selectively opened by using for instance targeted or programmable endonucleases. Although all nucleic acid molecules are still present in the reaction mixture, only those cleaved in the last opening reaction are able to be used in a subsequent (sequencing) process, for instance by ligating sequencing adapters to the open ends thereby selectively rendering these opened fragments ready for sequencing. Alternatively, the opened fragments may be degraded using exonuclease treatment, thereby enriching for the non-opened nucleic acid molecules for further processing. For instance, these non-opened molecules may be opened in a second round of selective opening using for instance programmable endonucleases targeted to these non-opened molecules.

The above mentioned approach has at least the following advantages.
The addition of barcodes to the ends of nucleic acid molecules after which samples can be pooled before further sample preparation steps are performed.
The need for a single CRISPR enzyme/guide complex for targeting a locus, in contrast to the usual use of two gRNAs per target locus.
The approach is in principle sequencing platform agnostic.
The approach can be used to target nucleic acid molecules without an amplification step, which enables the detection of native base modifications.
The mentioned approach can be applied to nucleic acid molecules of any length, i.e. short molecules (<1 Kbp) or long molecules (>5 Kbp).

Therefore in a first aspect, the invention pertains to an adapter comprising a protelomerase recognition sequence. Preferably, the adapter comprises a TeIN protelomerase recognition sequence. Preferably, the adapter is for use in a method of the invention. Preferably, the adapter can be linked to a nucleic acid molecule used in the method of the invention.

The adapter may be single-stranded. A single-stranded adapter preferably comprises a section, preferably at its 3′ end, that is capable of hybridizing to a nucleic acid molecule used in the method of the invention. The single-stranded adapter preferably can hybridize to a single-stranded overhang of the nucleic acid molecule, preferably a 3′ overhang of the nucleic acid molecule. The single-stranded part of the annealed single-stranded adapter may subsequently be filled in, i.e. is made double-stranded, using a polymerase, such as, but not limited to, Klenow (known by the skilled person to have 5′→3′ polymerase activity and 3′→5′ exonuclease activity but lacking 5′→3′ exonuclease activity) or a Bst-polymerase (known by the skilled person to be a DNA polymerase from Bacillus stearothermophilus having polymerase activity and strand displacement activity, but lacking 3′→5′ exonuclease activity). The filling-in step optionally results in the generation of a double-stranded protelomerase recognition sequence.

Preferably, the adapter is at least partly double-stranded. The at least partly double-stranded adapter may be ligated to a nucleic acid molecule in the method of the invention as defined herein. Preferably, at least 15%, 20%, 25%, 30%, 35%, 40%, 45%, 50%, 55%, 60%, 65%, 70%, 75%, 80%, 85%, 90%, 95% or 100% of the nucleotides in the adapter are double-stranded. Preferably, the protelomerase recognition sequence is double-stranded. The adapter may be 100% or “fully” double-stranded. The adapter may become fully double-stranded after ligation of the adapter to the nucleic acid molecule, e.g. by filing in the single-stranded part of the adapter using a DNA polymerase.

Preferably, the at least partly double-stranded adapter comprises two single-stranded molecules that may at least partly anneal to each other, i.e. the double-stranded adapter preferably comprises two open ends prior to ligating the adapter to the nucleic acid molecules as defined herein.

One end of the at least partly double-stranded adapter can be ligated to the nucleic acid molecule. Hence preferably at least the one end that is ligated to the nucleic acid molecule is double-stranded. The at least one end of double-stranded end of the adapter can be a blunt or a staggered or “sticky” end. Preferably, the adapter comprises at least one staggered end. Preferably, the end of the adapter that is ligated to the nucleic acid molecule has an end that is compatible with an end of the nucleic acid molecule. For example, in case the nucleic acid molecule comprises an end having an A-overhang, the adapter preferably comprises an end having a T-overhang. Similarly, in case the nucleic acid molecule is obtained by enzyme digestion leaving an overhang of 1, 2, 3, 4, 5 or more nucleotides, the adapter preferably comprises an overhang of respectively 1, 2, 3, 4, 5 or more nucleotides that are complementary to the overhang of the nucleic acid molecule.

The other end of the adapter preferably cannot be ligated to a nucleic acid molecule or an adapter. Any means to block ligation of an adapter end is suitable for use in the method of the invention. As a non-limiting example, the other end of the adapter may be single-stranded or comprises an incompatible overhang.

The adapter of the invention comprises a protelomerase recognition sequence, preferably a TeIN protelomerase recognition sequence. A protelomerase recognition sequence is any DNA sequence whose presence in a DNA template allows for its conversion into a closed linear DNA by the enzymatic activity of protelomerase. In other words, the protelomerase recognition sequence is required for the cleavage and religation of double stranded DNA by protelomerase to form covalently closed linear DNA. Typically, a protelomerase recognition sequence comprises a perfect palindromic sequence, i.e. a double-stranded DNA sequence having two-fold rotational symmetry.

The length of the perfect inverted repeat differs depending on the specific organism. In Borrelia burgdorferi, the perfect inverted repeat is 14 base pairs in length. In various mesophilic bacteriophages, the perfect inverted repeat is 22 base pairs or greater in length. Also, in some cases, e.g. E. coli N15, the central perfect inverted palindrome is flanked by inverted repeat sequences, i.e. forming part of a larger imperfect inverted palindrome.

A protelomerase recognition sequence as used in the invention preferably comprises a double stranded palindromic (perfect inverted repeat) sequence of at least 14 base pairs in length. Preferred perfect inverted repeat sequences include the sequences of SEQ ID NOs: 1-9 and variants thereof. SEQ ID NO: 1 (NCATNNTANNCGNNTANNATGN) is a 22 base consensus sequence. As e.g. disclosed in WO2010/086626, base pairs of the perfect inverted repeat are conserved at certain positions, while flexibility in sequence is possible at other positions. Thus preferably, SEQ ID NO: 1 is a minimum consensus sequence for a perfect inverted repeat sequence for use with a protelomerase in the process of the present invention. The protelomerase recognition sequence may have a sequence as described in WO2010/086626, which is incorporated herein by reference.

Preferably, the protelomerase recognition sequence has at least 80%, 85%, 90%, 95%, 96%, 97%, 98%, 99% or 100% sequence identity with SEQ ID NO: 10. The sequence of SEQ ID NO: 10 is:

### 5′-TATCAGCACACAATTGCCCATTATACGCGCGTATAATGGACTATTGTGTGCTGATA-3′.

Preferably, the protelomerase cleaves the adapter sequence between positions 28-29 in the recognition sequence and closes the cleaved ends.

The adapter may consists of the protelomerase recognition sequence. Alternatively, the adapter may comprise additional nucleotides. The adapter may comprise an identifier sequence or “barcode” or “tag”. The identifier is preferably at least one of a sample identifier and an UMI. Preferably, the recognition sequence remains part of the nucleic acid molecule after cleaving and closing the cleaved ends.

The UMI may be a separate sequence within the adapter or, in case the protelomerase recognition sequence comprises degenerate nucleotides, these degenerate nucleotides may be used to introduce an identifier. For instance, in case of degenerate nucleotides in the protelomerase recognition sequence for one sample an adapter may be used with one or more specific nucleotides within this recognition sequence, whereas for a second or further sample, other specific nucleotides are used at this position, thereby creating an identifier sequence within the protelomerase recognition sequence. The adapter may comprise and sample identifier as well as an UMI.

A sample identifier may connect the sequence of a nucleic acid molecule to a specific sample. For example, the adapters used in the method of the invention may comprise an identifier sequence that is specific for a certain sample. Each additional sample can be processed using adapters having an identifier sequence specific for said additional sample. The processed samples can subsequently be pooled and the obtained sequences can be assigned to a specific sample using the sample identifier sequence.

A UMI is a substantially unique sequence or barcode, preferably fully unique, that is specific for a nucleic acid molecule, i.e. unique for each nucleic acid molecule used in the method of the invention. The UMI may have random, pseudo-random or partially random, or non-random nucleotide sequences. A UMI can be used to uniquely identify the originating molecule from which a sequencing read is derived. For example, reads of amplified nucleic acid molecules can be collapsed into a single consensus sequence from each originating nucleic acid molecule. As indicated above, the UMI may be fully or substantially unique. Fully unique is to be understood herein as that every adapter-ligated nucleic acid molecule provided in the method of the invention comprises a unique tag that differs from all the other tags comprised in further adapter-ligated nucleic acid molecules used in the method of the invention. Substantially unique is to be understood herein in that each adapter-ligated nucleic acid molecule provided in the method of the invention comprises a random UMI, but a low percentage of these adapter-ligated nucleic acid molecules may comprise the same UMI. Preferably, substantially unique molecular identifiers are used in case the chances of tagging the exact same molecule comprising the same sequence with the same UMI is negligible. Preferably, the UMI is fully unique in relation to a specific sequence of the nucleic acid molecule. The UMI preferably has a sufficient length to ensure this uniqueness. In some implementations, a less unique molecular identifier (i.e. a substantially unique identifier, as indicated above) can be used in conjunction with other identification techniques to ensure that each nucleic acid molecule is uniquely identified during the sequencing process.

An identifier sequence may range in length from about 2 to 100 nucleotide bases or more, and preferably has a length between about 4-16 nucleotide bases. The identifier sequence can be a consecutive sequence or may be split into several subunits. Each of these subunits. These subunits may be present in a single adapter or may be present in separate adapters. For instance, if the nucleic acid molecule is flanked by two adapters, each of these two adapters may comprise a subunit of the identifier sequence. In order to obtain consensus sequences, the sequence reads obtained in the method of the invention may be grouped based on the information each of the two subunits.

Preferably the identifier sequence does not contain two or more consecutive identical bases. Furthermore, there is preferably a difference between identifier sequences of at least two, preferably at least three bases.

Means for designing and constructing an adapter for use in the invention are well known to the skilled person and the invention is not limited to any particular adapter design and/or construction. As a non-limiting example, two oligonucleotides can be constructed and annealed to one another under controlled conditions, resulting in at least partly double-stranded adapter for use in the invention. As a further non-limiting example, a long and a short oligonucleotide can be constructed, wherein the short oligonucleotide can anneal to the end of the long oligonucleotide. Preferably at least 60%, 65%, 70%, 75%, 80%, 85%, 90%, 95% or 100% of the nucleotides of the short oligonucleotide can anneal to the long oligonucleotide. Preferably the short oligonucleotide is 100% complementary to a section of the long oligonucleotide. Preferably this complementary section is located 3′ of the protelomerase recognition sequence, e.g. 1, 2, 3, 4, 5, 6, 7, 8, 9, 10 or more nucleotides 3′ of the recognition sequence. The complementary section may be located in between the protelomerase recognition sequence and the 3′ end of the long oligonucleotide. The complementary section may be located at the 3′ end of long oligonucleotide. Alternatively, the complementary section may be located upstream of the 3′ end of the long oligonucleotide, e.g. at least 1, 2, 3, 4, 5, 6, 7, 8, 9, 10, 15 or more nucleotides upstream of the 3′ end of the long oligonucleotide. After annealing the short and long oligonucleotide, the part of the long oligonucleotide located 5′ of the complementary section may be filled in, thus producing a double-stranded adapter, wherein the double-stranded adapter may have 3′ overhang, wherein the 3′ overhang is the 3′ end of the long oligonucleotide. Filling in the single-stranded sequence, i.e. to generate a double-stranded sequence can be done using any conventional polymerase, such as, but not limited to Klenov or BST-polymerase. A preferred polymerase is a BST-polymerase.

Optionally, the adapter of the invention further comprises a restriction enzyme recognition site between the protelomerase recognition sequence and the part of the adapter for ligation to the nucleic acid molecule.

In a further aspect, the invention pertains to a method for preparing a nucleic acid molecule library. Preferably, the method comprises one or more of the following steps:
a) providing a sample comprising at least a first and a second nucleic acid molecule, wherein the first nucleic acid molecule comprises a first target sequence not present in the second nucleic acid molecule;
b) ligating an adapter as defined herein, i.e. comprising a protelomerase recognition sequence, to the ends of the first nucleic acid molecule to provide an adapter ligated nucleic acid molecule;
c) contacting the adapter ligated nucleic acid molecule with a protelomerase to cleave and covalently close the cleaved ends, resulting in a first nucleic acid molecule comprising closed ends; and
d) cleaving the first nucleic acid molecule comprising the closed ends to provide a first nucleic acid comprising one open end and one closed end.

Optionally, no adapters comprising protelomerase recognitions sequences are ligated to the ends of the second nucleic acid molecule, or amplicons thereof. Within such embodiment, the second nucleic acid molecules are eliminated, e.g. by exonuclease treatment between step c and d. Selective adapter ligation to a specific nucleic acid molecule may be achieved by creating specific ends, suitable for selective adapter ligation in step b, at the first nucleic acid molecule, which specific ends are not created at the ends of the second nucleic acid molecule. For instance, specific staggered ends may be created by a specific endonuclease capable of creating such staggered ends, such as, but not limited to, a type V CRISPR endonuclease such as Cpf1 in combination with a first crRNA targeted to a sequence upstream of the first target sequence and a second crRNA targeted to a sequence downstream of the first target sequence. Within such embodiment, the adapters used in step b should, at their side for ligation to the first nucleic acid molecule, comprise an overhang compatible for ligation to the staggered ends so created. Within this embodiment, the closed first nucleic acid molecule may be opened in step d by cleavage at a specific sequence within the adapter. For instance, if adapters are used that comprise a particular restriction enzyme recognition site between the side for ligation and the protelomerase recognition sequence. Alternatively, the closed first nucleic acid molecule may be opened in step d by cleavage at a sequence within the first nucleic acid molecule, such as the first target sequence.

Alternatively, in step b of the method of the invention, adapters are ligated to both the first and second nucleic acid molecules. Within such an embodiment, the closed second nucleic acid molecule obtained in step c may to be eliminated specifically from the reaction mixture comprising the closed first nucleic acid molecule prior to step d. This may be achieved by cleaving the closed second nucleic acid molecule at a specific sequence, i.e. a second target sequence, not present in the closed first nucleic acid molecule. Within such embodiment the second nucleic acid molecule of the method as defined herein comprises a second target sequence that is not present in the first nucleic acid molecule. The subsequent opened second nucleic acid molecule can be eliminated by exonuclease treatment. As the second nucleic acid molecule is now absent, the closed first nucleic acid may be opened in a specific or aspecific manner, for instance by cleaving at a sequence within the adapter as indicated herein above or at a sequence present in the first nucleic acid molecule. In case the method is such that the closed second nucleic acid molecule is not eliminated prior to step d, this closed second nucleic acid molecule is still present in the reaction mixture comprising the closed first nucleic acid molecule in step d. In such a design, the first nucleic acid is preferably selectively opened by cleaving at the first target sequence not present in the second nucleic acid molecule. Such a method preferably comprises the following steps:
a) providing a sample comprising at least a first and a second nucleic acid molecule, wherein the first nucleic acid molecule comprises a first target sequence not present in the second nucleic acid molecule and wherein optionally the second nucleic acid molecule comprises a second target sequence;
b) ligating an adapter as defined herein, i.e. comprising a protelomerase recognition sequence, to the ends of the first and second nucleic acid molecule to provide adapter ligated nucleic acid molecules;
c) contacting the adapter ligated nucleic acid molecules with a protelomerase to cleave and covalently close the cleaved ends, resulting in a first and second nucleic acid molecule comprising closed ends; and
d) cleaving the first nucleic acid molecule comprising the closed ends at the first target sequence, to provide a first nucleic acid comprising one open end and one closed end.
   A preferred protelomerase is a TeIN protelomerase.

It is to be understood herein that an effective number of components is used in the method of the invention. A nucleic acid molecule library prepared by the method of the invention is preferably suitable for further processing of the nucleic acid molecule such as, but not limited to, cloning, amplification, sequencing and the like. Hence in additional aspects, the invention also concerns a method for cloning a nucleic acid molecule library, a method for amplifying a nucleic acid molecule library or a method for sequencing a nucleic acid molecule library, using the steps as described herein.

Preferably, the prepared nucleic acid molecule library is enriched for a nucleic acid molecule comprising a sequence of interest. “Enriched” is understood herein to mean a reduction or elimination of nucleic acid molecules not having a sequence of interest, either by (i) selective exclusion of nucleic acid molecules not having a sequence of interest from further processing steps, or by (ii) selective inclusion of nucleic acid molecules having a sequence of interest for further processing steps. The selectively excluded nucleic acid molecules may be degraded, e.g. by exonuclease treatment. The selectively included nucleic acid molecules may e.g. be cloned, amplified and/or sequenced.

The prepared nucleic acid library preferably comprises nucleic acid molecules having one closed end and one open end.

In an embodiment, the method as defined herein comprises a step a) of providing a sample comprising at least a first and a second nucleic acid molecule. Preferably the first nucleic acid molecule comprises a first target sequence not present in the second nucleic acid molecule. Preferably, the second nucleic acid molecule comprises a second target sequence. Optionally, the second target sequence is also present in the first nucleic acid molecule. Alternatively, the second target sequence is not present in the first nucleic acid molecule.

Preferably, the first nucleic acid molecule comprises a sequence of interest and the second nucleic molecule does not comprise said sequence of interest. In this embodiment, the first nucleic acid molecule will be present in the prepared nucleic acid molecule library and will preferably be processed further.

In an alternative embodiment, the first nucleic acid molecule does not comprise a sequence of interest, but the second nucleic acid molecule comprises said sequence of interest. In this embodiment, the second nucleic acid molecule will be present in the prepared nucleic acid molecule library and will preferably be processed further.

The sample comprising at least a first and a second nucleic acid molecule may be from any source, e.g. human, animal, plant, microorganism, and maybe of any kind, e.g. endogenous or exogenous to the cell, for example genomic DNA, chromosomal DNA, artificial chromosomes, plasmid DNA, or episomal DNA, cDNA, RNA, mitochondrial, or of an artificial library such as a BAC or YAC or the like. The DNA may be nuclear or organellar DNA. Preferably, the DNA is chromosomal DNA, preferably endogenous to the cell. Preferably, the first, second and optionally further nucleic acid molecules present in the sample that is used as starting material for the method of the invention is any one of DNA, such as genomic DNA, chromosomal DNA, organellar DNA, mitochondrial DNA, artificial chromosomes, plasmid DNA, episomal DNA, cDNA and RNA.

The first and second nucleic acid molecules may be long nucleic acid molecules, provided e.g. by cell lysis and optionally lysis of an organelle. The nucleic acid molecules used in the method of the invention may have a size of at least about 50 kb, 100 kb, 150 kb, 200 kb, 300 kb, 400 kb, 500 kb, 600 kb, 700 kb, 800 kb, 900 kb or at least about 1000 kb (1 Mb). The first and/or second nucleic acid for use in the invention may be high molecular weight (HMW) nucleic acids or ultra-high molecular weight (uHMW) nucleic acids. uHMW nucleic acids may have a length of at least 1 Mb. The nucleic acid molecules used in the method of the invention may have a size of at least 1.1 Mb, 1.3 Mb, 1.5 Mb, 1.7 Mb, 2 Mb, 2.5 Mb, 3 Mb, 4 Mb, 5 Mb, 6 Mb, 7 Mb, 8 Mb, 9 Mb or at least about 10 Mb.

Alternatively long nucleic acid molecules may first be fragmented, resulting in a first and second nucleic acid molecule. Therefore in an embodiment, the first and second nucleic acid molecules in step a) are provided by fragmentation. The fragmentation is preferably the fragmentation of a genomic nucleic acid molecule.

The skilled person is familiar with means to fragment longer nucleic acid molecules and the invention is not limited to any specific means for fragmenting the longer nucleic acid molecule. The fragmented nucleic acids are preferably fragmented genomic DNA. DNA, and in particular genomic DNA, can be fragmented using any suitable method known in the art. Methods for DNA fragmentation include, but are not limited to, enzymatic digestion and mechanical force.

Non-limited examples of fragmenting the nucleic acid molecule using mechanical force include the use of acoustic shearing, nebulization, sonication, point-sink shearing, needle shearing and French pressure cells.

Enzymatic digestion for fragmenting a nucleic acid molecule, which molecule comprises at least one of the first and second nucleic acid molecule as defined herein, includes, but is not limited to, endonuclease restriction. Enzymatic digestion, such as e.g. used in AFLP® technology, may further result in a complexity reduction of the nucleic acid sample. The skilled person knows which enzymes to select for the DNA fragmentation. As a non-limiting example, at least one frequent cutter and at least one rare cutter can be used for the fragmentation of the nucleic acid sample. A frequent cutter preferably has a recognition site of about 3-5 bp, such as, but not limited to MseI. A rare cutter preferably has a recognition site of >5 bp, such as but not limited to EcoRI.

In certain embodiments, in particular when the sample contains or is derived from a relative large genome, it may be preferred to use a third enzyme, rare or frequent cutter, to obtain a larger set of restriction fragments of shorter size.

The method of the invention is not limited to any specific restriction endonuclease. The endonuclease may be a type II endonuclease, such as EcoRI, Msel, Pstl etc. In certain embodiments a type IIS or type III endonuclease may be used, i.e. an endonuclease of which the recognition sequence is located distant from the restriction site, such as, but not limited to, Acelll, Alwl, AlwXl, Alw26l, Bbvl, Bbvll, Bbsl, Bed, Bce83l, Bcefl, Bcgl, Binl, Bsal, Bsgl, BsmAl, BsmFl, BspMl, Earl,Ecil, Eco3ll, Eco57l, Esp3l, Faul, Fokl, Gsul, Hgal, HinGUll, Hphl, Ksp632l, Mboll, Mmel, Mnll, NgoVlll, Plel, RleAl, Sapl, SfaNl, TaqJl and Zthll III. Restriction fragments can be blunt-ended or have protruding ends, depending on the endonuclease used.

In a preferred embodiment, the recognition site of at least one of the frequent cutter and the rare cutter is within or in close proximity of the sequence of interest, e.g. the recognition site of the frequent cutter or the rare cutter is located about 0-10000, 10-5000, 50-1000 or about 100-500 bases from the sequence of interest.

The current method as disclosed herein can also be used in AFLP® technology, e.g. for polyploid cells. The AFLP® technology is e.g. described in more detail in WO2007/114693, WO2006/137733 and WO2007/073165, which are incorporated herein by reference. The AFLP® technology as described in the art can be modified by attaching an adapter comprising a protelomerase recognition sequence as described herein, to the restricted nucleic acid sample.

In addition or alternatively, the nucleic acid sample may be digested using a programmable nuclease, preferably using at least one of a CRISPR nuclease, a zinc finger nuclease, TALENs and meganucleases.

Optionally, the first and/or second nucleic acid molecule may be modified to comprise an A-tail, preferably to facilitate ligation to the partly, or fully, double-stranded adapter comprising a protelomerase recognition sequence and further comprising a T-overhang. Hence prior to annealing an adapter to the fragmented nucleic acid, the method of the invention may optionally comprise a step of A-tailing the fragmented nucleic acid sample. A-tailing reactions are well-known in the art and the skilled person straightforwardly understands how to perform an A-tailing reaction, such as e.g. using a Klenow fragment (exo-).

The nucleic acid sample comprising at least one of a first and a second nucleic acid molecule, may comprise a plurality of further nucleic acid molecules. Hence in some embodiments, the nucleic acid sample comprises only a first nucleic acid molecule and only a second nucleic acid molecule. In other embodiments, the nucleic acid sample comprises a first nucleic acid molecule, a second nucleic acid molecule, in addition to a plurality of other nucleic acid molecules. Preferably, said further nucleic acid molecules do not comprise a first target sequence. Optionally, the further nucleic acid molecules do not comprise a second target sequence. This plurality of other nucleic acid molecules may be derived from at least one of the same organism, the same tissue, the same cell, the same organelle and/or the same molecule from which the first and second nucleic acid molecules are derived.

It is understood herein that a nucleic acid sample comprising a first nucleic acid molecule may also include a nucleic acid sample comprising a plurality of first nucleic acid molecules. Similarly, it is understood herein that a nucleic acid sample comprising a second nucleic acid molecule may also include a nucleic acid sample comprising a plurality of second nucleic acid molecules. Preferably, the first nucleic acid molecule is derived from the same organism, the same tissue, the same cell, the same organelle and/or the same molecule from which the second nucleic acid molecule is derived. The first and second nucleic acid molecule may have essentially the same sequence, with the exception of one or more nucleotides. As a non-limiting example, the first and second nucleic acid molecule may be allele variants. Alternatively, the first and second nucleic acid molecules may be very dissimilar, e.g. have less than 40%, 30%, 20%, 10% or 5% sequence identity. A predominant difference between the first and second nucleic acid nucleic acid molecule used in the invention, is that the first nucleic acid molecule comprises a target sequence that is not present in the second nucleic acid molecule.

Optionally, the second nucleic acid molecule may comprise a second target sequence. This second target sequence may or may not also be present in the first nucleic acid molecule.

In an embodiment, the method comprises a step b) of ligating an adapter to the ends of the first and second nucleic acid molecule to provide adapter ligated nucleic acid molecules. The adapter is preferably an adapter as defined herein, i.e. an adapter comprising a protelomerase recognition sequence. The adapter is preferably ligated to both ends of the first nucleic acid molecule and both ends of the second nucleic acid molecule. Preferably, the adapter is ligated to both ends of at least 80%, 85%, 90%, 95%, 96%, 97%, 98%, 99% or 100% of the nucleic acids present in the sample. Preferably after the ligation step, all nucleic acid molecules in the sample comprise an adapter on both ends. Put differently preferably all, or substantially all, nucleic acids in the sample are flanked on both sites by a covalently linked adapter. Ligation of an adapter can be performed using any conventional method known to the skilled person and the invention is not limited to any specific ligation method or ligation enzyme (ligase). Preferably to facilitate the ligation, the adapter comprises an end that is compatible to the end of the nucleic acid molecules, e.g. by using nucleic acid molecules obtained through the use of restriction endonucleases and compatible staggered ends on the adapters.

In an embodiment, the fragmented nucleic acid molecules may be polished to create blunt ends, followed by the addition of a 3′-A staggered overhang. The polishing step may be performed using any conventional means known in the art. Similarly, the addition of a 3′-A overhang may be achieved using any conventional method known to the skilled person. The nucleic acid molecules comprising a 3′-A-overhang may subsequently be ligated to compatible adapters comprising a 5′-T-overhang.

In an embodiment, the step of fragmentation and adapter ligation may be combined in a single step, e.g. by means of tagmentation. In this embodiment, the adapter in step b) is ligated by tagmentation, preferably using a Tn5 transposase. Transposases randomly cut the long DNA molecules in shorter nucleic acid molecules and adapters can be ligated on either side of the cleaved points. Tagmentation or “transposase mediated fragmentation and tagging” is a process that is well-known for the person skilled in the art, for example as exemplified in the workflow for Nextera™. The adapters may comprise sequences that make them compatible for use in a tagmentation reaction. Preferably, the adapters used in a tagmentation reaction further comprise a transposase sequence. The transposase sequence is preferably compatible with the transposase used in the tagmentation reaction. The tagmentation reaction may be followed by a repair step to ensure that all, or substantially all, generated nucleic acid molecules comprise an adapter on both sides. Hence the nucleic acid molecules comprising ligated adapters, optionally obtained by tagmentation, may be repaired to remove any single-stranded breaks. Preferably, the repair step takes place prior to contacting the molecules with a TeIN protelomerase in step c). Such repair step can be performed using any conventional means known in the art.

Optionally, the protelomerase recognition sequence is attached to the nucleic acid molecules via a primer instead of an adapter. Preferably said primer comprises
i) a 3′-end for annealing to a primer binding site present in at least the first and/or second nucleic acid molecule, or to an, optionally universal, primer binding site in an adapter that has been ligated to said at least first and/or second nucleic acid molecule; and
ii) a protelomerase recognition site in a 5′-tail of such primer.
   Optionally, the primer binding site is a unique sequence, i.e. a sequence that is only present in the first and/or second nucleic acid molecule. Using one or more of these primers, the protelomerase sequence may be introduced in amplicons produced via PCR using the first and/or second nucleic acid molecule as template. Within such embodiment, in step b) instead of ligating an adapter as defined herein to the ends of the first nucleic acid molecule and optional second nucleic acid molecule to provide an adapter ligated nucleic acid molecules, the first and optional second nucleic acid molecules are amplified using at least one primer comprising a protelomerase recognition site; the subsequent steps are then performed on the resulting amplicons which can be closed upon protelomerase treatments. Alternatively, the protelomerase sequence may be introduced via a single step of denaturation, annealing of the primer and filling in the single strand overhang.

In those embodiments wherein the adapter is attached via a primer or tagmentation, instead of ligation via a (partly) double-stranded adapter, the terms “ligating” or “ligation” as used herein may be thus be replaced for the terms “attaching” or “attachment”.

In an embodiment, the method of the invention comprises a step c) of contacting the adapter ligated nucleic acid molecules with a protelomerase to cleave and covalently close the cleaved ends, resulting in a first and a second nucleic acid molecule comprising closed ends. Preferably, the protelomerase is a TeIN protelomerase.

Preferably, the first nucleic acid molecule comprises an adapter on both ends (i.e. at the 5′ and 3′ end) of the molecule and the second nucleic acid molecule comprises and adapter on both ends of the molecule, wherein said adapters have a protelomerase recognition sequence. Contacting the first and the second molecule comprising the adapters with a protelomerase under suitable conditions results in the cleavage, or “restriction” of the adapters. Simultaneously, the protelomerase can covalently close the nucleic molecules, resulting in a closed first nucleic acid and a closed second nucleic acid. Closed linear DNA molecules typically comprise covalently closed ends resulting in protection of terminal nucleotides against loss or damage.

A preferred protelomerase for use in the invention is a bacteriophage protelomerase. A protelomerase can be selected from the group consisting of:phiHAP-1 from Halomonas aquamarina, PY54 from Yersinia enterolytica, phiKO2 from Klebsiella oxytoca, VP882 from Vibrio sp. and NI 5 from Escherichia coli, or variants of any thereof. The protelomerase may have an amino acid sequence as disclosed in WO2010/086626, which is incorporated herein by reference. The use of bacteriophage NI 5 (TeIN) protelomerase or a variant thereof is particularly preferred. A preferred protelomerase has a sequence of at least 80%, 85%, 90%, 95%, 96%, 97%, 98%, 99% or 100% sequence identity with SEQ ID NO: 11. Variants include homologues or mutants thereof. Mutants include truncations, substitutions or deletions with respect to the native sequence. A variant preferably produces closed linear DNA from a template comprising a protelomerase recognition sequence as described herein above.

The method may optionally comprise a step c1) of exposing the sample to an exonuclease after obtaining the nucleic acid molecules comprising closed ends in step c) and prior to cleaving the first nucleic acid molecule comprising the closed ends in step d). Hence in an embodiment, the method of the invention comprises the steps of:
a) providing a sample comprising at least a first and a second nucleic acid molecule, wherein the first nucleic acid molecule comprises a first target sequence not present in the second nucleic acid molecule and wherein optionally the second nucleic acid molecule comprises a second target sequence;
b) ligating an adapter as defined herein, i.e. comprising a protelomerase recognition sequence, to the ends of the first and second nucleic acid molecule to provide adapter ligated nucleic acid molecules;
c) contacting the adapter ligated nucleic acid molecules with a protelomerase to cleave and covalently close the cleaved ends, resulting in a first and second nucleic acid molecule comprising closed ends;
c1) exposing the sample comprising the first and second nucleic acid molecule comprising closed ends to an exonuclease; and
d) cleaving the first nucleic acid molecule comprising the closed ends at the first target sequence, to provide a first nucleic acid comprising one open end and one closed end.

The exonuclease may digest any nucleic acid molecule not comprising two closed ends, i.e. comprising one or two open ends. Such nucleic acid molecules are for example, but not limited to, nucleic acid molecules without adapters, nucleic acid molecules with one or two adapters having an open end, and/or cleaved nucleic acid molecules having one open end and one closed end.

The nucleic acid molecules having two closed ends are protected from degradation, while the non-protected fragments are degraded, resulting in enrichment or complexity reduction of the nucleic acid molecules comprising the sequence of interest, i.e. the first or optionally the second nucleic acid molecule. Therefore in an embodiment, the method of the invention takes the approach of removal of an undesired (non-target) part of the nucleic acid sample. As a non-limiting example, the adapters in step b) may be ligated to nucleic acid molecules having a selective staggered overhang, for example created by enzymatic digestion. The molecules comprising the adapters are subsequently closed in step c), and the exonuclease treatment in step c1) may digest any nucleic acid molecule not having two closed ends. The exonuclease treatment in step c1) may thus result in an enrichment of nucleic acid molecules comprising closed ends.

The exonuclease may be exonuclease I, III, V, VII, VIII, or related enzyme, or any combination thereof. Exonuclease III recognizes nicks and extend the nick to a gap until a piece of ssDNA is formed. Exonuclease VII can degrade this ssDNA. Exonuclease I also degrades ssDNA. ExoIII and ExoVII is a preferred combination of exonucleases for use in step c) of the method of the invention.

Exonuclease V is capable of degrading ssDNA and dsDNA in both 3′ to 5′ and in 5′ to 3′ direction. Therefore in a preferred embodiment, the exonuclease in step c) of the method of the invention is an exonuclease that is capable of degrading ssDNA and dsDNA in both 3′ to 5′ and in 5′ to 3′ direction, preferably an exonuclease V.

Further information on methods for degrading non-target sequences is provided in U.S. Patent Publication No. 2014/0134610, which is incorporated herein by reference in its entirety for all purposes.

Step c1) is preferably performed at conditions (e.g. time, temperature, enzyme concentration) sufficient for the exonucleases to degrade substantially all non-protected fragments. Preferably, step c1) is performed at conditions and time sufficient for the exonucleases to degrade all non-protected fragments. Step c1) is preferably performed for about 1 minute to about 12 hours, preferably 30 min, at about 10-90° C., preferably about 37° C.,

After step c1), the exonuclease may be inactivated by, for example, but not limited to, at least one of a Proteinase, e.g. Proteinase K, treatment or heat inactivation. Such techniques are standard in the art and the skilled person straightforwardly understands how to inactivate an exonuclease. A preferred inactivation step is heating the sample at a temperature of about 50-90° C., preferably about 75° C., for about 1-120 minutes, preferably about 10 minutes.

In an embodiment, the method of the invention, comprises a step d) of cleaving the first nucleic acid molecule comprising the closed ends at the first target sequence, to provide a first nucleic acid comprising one open end and one closed end. “Cleaving” is understood herein the generation of a double-stranded break. The double-stranded break may be created by the use of a nuclease or by the use of two nickases that cleave opposite stands. The double stranded break may create a blunt open end of the first, and optionally second, nucleic acid molecule. After cleavage the cleaved nucleic acid molecule may thus have one open blunt end and one closed end. Alternatively, the double stranded break may create a staggered open end of the cleaved nucleic acid molecule. After cleavage, the cleaved nucleic acid molecule may thus have one open staggered end and one closed end.

Preferably, the first nucleic acid molecule in step d) is cleaved by a programmable nuclease or a restriction endonuclease. The first nucleic acid molecule thus comprises a target sequence that is not present in the second nucleic acid molecule. The first nucleic acid molecule may comprise the target sequence more than once, e.g. the first nucleic acid molecule may comprise the target sequence 1, 2, 3, 4, 5, 6 or more times. In an embodiment, the second nucleic acid molecule may comprise a target sequence that is not present in the first nucleic acid molecule. The second nucleic acid molecule may comprise the target sequence more than once, e.g. the second nucleic acid molecule may comprise the target sequence 1, 2, 3, 4, 5, 6 or more times.

The skilled person easily understands that this step can be extended to additional nucleic acid molecules, e.g. at least a third, fourth or fifth or further nucleic acid molecule. Each nucleic acid molecule may optionally comprise a target sequence that is absent in any of the other nucleic acid molecules.

It is thus understood herein that the nucleic acid sample comprises at least one nucleic acid molecule comprising a sequence of interest, i.e. the first nucleic acid molecule as defined herein or optionally the second nucleic acid molecule as defined herein. Put differently, the nucleic acid sample thus may comprise 1, 2, 3, 4, 5, 6, 7, 8, 9, 10 or more sequences of interest, such as at least about 50, 100, 150, 200, 250, 300, 350, 400, 450, 500, 750, 1000 or more sequences of interest, wherein preferably each sequences of interest within the sample has a distinct target sequence. The method of the invention may provide for a simultaneous enrichment of these sequences of interest from a nucleic acid sample. Therefore optionally, in step d) of the method of the invention, multiple gRNA-CAS complexes are added for enrichment of nucleic acid molecules from a nucleic acid sample. Preferably, these multiple gRNA-CAS complexes may comprise the same CRISPR-nuclease, but may differ in their gRNA. For example, for each nucleic acid molecule comprising a sequence of interest, a distinct gRNA molecule may be used. For e.g. at least about 50, 100, 150, 200, 250, 300, 350, 400, 450, 500, 750, 1000 or more nucleic acid molecules, preferably at least about 50, 100, 150, 200, 250, 300, 350, 400, 450, 500, 750, 1000 or more gRNA molecules may be used in the method of the invention.

The first, and optionally second, nucleic acid molecule comprising closed ends may be cleaved by a restriction endonuclease. In an embodiment wherein the first and second nucleic acid molecule are cleaved, the first and second nucleic acid molecule are cleaved by a different endonuclease. Any sequence-specific endonuclease may be suitable for use in the invention. The endonuclease may be a so-called “restriction endonuclease” or “restriction enzyme”, e.g. a Type I, Type II, Type III, Type IV or Type V restriction endonuclease. A preferred restriction endonuclease is a Type II restriction endonuclease, preferably Type IIP or Type IIS. In case a fragmentation in step a) is performed by cleaving the DNA with a restriction enzyme, the enzyme used in step d) is preferably a different endonuclease.

The first nucleic acid molecule, and optionally the second nucleic acid molecule, may be cleaved by a programmable nuclease. In an embodiment wherein the first and second nucleic acid molecule are cleaved, the first and second nucleic acid molecule are cleaved by a different programmable nuclease, i.e. programmable nucleases that recognize different target sequences. A programmable nuclease may be selected from the group consisting of a zinc finger nuclease, a meganuclease, a TAL-effector nuclease and an RNA-guided CRISPR nuclease. Preferably, the programmable nuclease is an RNA-guided CRISPR (Clustered Regularly Interspaced Short Palindromic Repeats) nuclease.

The RNA-guided CRISPR nuclease is preferably part of a gRNA-Cas complex. A gRNA-CAS complex is to be understood herein as a CRISPR associated (CAS) protein, or CRISPR-nucleases, complexed with a guide RNA. A CRISPR-nuclease comprises a nuclease domain and at least one domain that interacts with a guide RNA. When complexed with a guide RNA, the CRISPR-nuclease is directed to the target sequence by a guide RNA. The guide RNA interacts with the CRISPR-nuclease as well as with the target sequence, such that, once directed to the site comprising the specific target sequence via the guide sequence, the CRISPR-nuclease is able to introduce a break at the target sequence. Preferably, the CRISPR-nuclease is able to introduce a single or double strand break at the target sequence, in case one or both domains of the nuclease are catalytically active, respectively. The skilled person is well aware of how to design a guide RNA in a manner that it, when combined with a CRISPR-nuclease, effects the introduction of a single- or double-stranded break at a predefined target site in the first, and/or optionally second, nucleic acid molecule.

CRISPR-nucleases can generally be categorized into six major types (Type I-VI), which are further subdivided into subtypes, based on core element content and sequences (Makarova et al, 2011, Nat Rev Microbiol 9:467-77 and Wright et al, 2016, Cell 164(1-2):29-44). In general, the two key elements of a CRISPR-CAS system complex is a CRISPR-nuclease and a crRNA. CrRNA consists of short repeat sequences interspersed with spacer sequences derived from invader DNA. CAS proteins have various activities, e.g., nuclease activity. Thus, gRNA-CAS complexes provide mechanisms for targeting a specific sequence as well as certain enzyme activities upon the sequence.

Type I CRISPR-CAS systems typically comprise a Cas 3 protein having separate helicase and DNase activities. For example, in the Type 1-E system, crRNAs are incorporated into a multi-subunit effector complex called Cascade (CRISPR-associated complex for antiviral defense) (Brouns et al, 2008, Science 321 : 960-4), which specifically binds to duplex DNA and triggers degradation by the Cas3 protein (Sinkunas et al., 2011, EMSO J 30: 1335-1342; Beloglazova et al., 2011, EMBO J 30:616-627).

Type II CRISPR-CAS systems include a signature Cas9 protein, a single protein (about 160 KDa), capable specifically cleaving duplex DNA. The Cas9 protein typically contains two nuclease domains, a RuvC-like nuclease domain near the amino terminus and the HNH (or McrA-like) nuclease domain near the middle of the protein. Each nuclease domain of the Cas9 protein is specialized for cutting one strand of the double helix (Jinek et al, 2012, Science 337 (6096): 816-821). The Cas9 protein is an example of a CAS protein of the type II CRISPR/-CAS system and forms an endonuclease, when combined with the crRNA and a second RNA termed the trans-activating crRNA (tracrRNA), which targets the invading pathogen DNA for degradation by the introduction of DNA double strand breaks (DSBs) at the position in the pathogen genome defined by the crRNA. Jinek et al. (2012, Science 337: 816-820) demonstrated that a single chain chimeric guide RNA (a sgRNA) produced by fusing an essential portion of the crRNA and tracrRNA was able to form a functional endonuclease in combination with the Cas9 protein.

Type III CRISPR-CAS systems contain polymerase and RAMP modules. Type III systems can be further divided into sub-types III-A and III-B. Type III-A CRISPR-CAS systems have been shown to target plasmids, and the polymerase-like proteins of Type III-A systems are involved in the specific cleavage of DNA (Marraffini and Sontheimer, 2008, Science 322: 1843-1845). Type III-B CRISPR-CAS systems have also been shown to target RNA (Hale et al, 2009, Cell 139:945-956).

Type IV CRISPR-CAS systems include Csf1, an uncharacterized protein proposed to form part of a Cascade-like complex, though these systems are often found as isolated cas genes without an associated CRISPR array.

A Type V CRISPR-CAS system has recently been described, the Clustered Regularly Interspaced Short Palindromic Repeats from Prevotella and Francisella 1 or CRISPR/Cpf1. Cpf1 genes are associated with the CRISPR locus and coding for an endonuclease that use a crRNA to target DNA. Cpf1 is a smaller and simpler endonuclease than Cas9, which may overcome some of the CRISPR-Cas9 system limitations. Cpf1 is a single RNA-guided endonuclease lacking tracrRNA, and it utilizes a T-rich protospacer-adjacent motif. Cpf1 cleaves DNA via a staggered DNA double-stranded break (Zetsche et al (2015) Cell 163 (3): 759-771). The type V CRISPR-CAS system preferably includes at least one of Cpf1, C2c1 and C2c3.

A Type VI CRISPR-CAS system may comprise a Cas13a protein, which comprises RNaseA activity. In case the target nucleic acid fragment is RNA, the at least first and second gRNA-CAS complex of the method of the invention may comprise Cas13a, such as, but not limited to Cas13 a from Leptotreichia wadee (LwCas13a) or from Leptotrichia shahii (LshCas13a) such as described in Gootenberg et al., Science. 2017 Apr 28; 356(6336):438-442.

The gRNA-CAS complex of the method of the invention may comprise any CRISPR-nuclease as defined herein above. Preferably, the gRNA-CAS complex used in the method of the invention comprises a Type II CRISPR-nuclease, e.g., Cas9 (e.g., the protein of SEQ ID NO: 12, encoded by SEQ ID NO: 13, or the protein of SEQ ID NO: 14) or a Type V CRISPR-nuclease, e.g. Cpf1 (e.g., the protein of SEQ ID NO: 15, encoded by SEQ ID NO: 16) or Mad7 (e.g. the protein of SEQ ID NO: 17 or 18), or protein derived thereof, having preferably at least about 70%, 80%, 90%, 91%, 92%, 93%, 94%, 95%, 96%, 97%, 98%, or 99% sequence identity to said protein over its whole length.

Preferably, the gRNA-CAS complex of the method of the invention comprises a Type II CRISPR-nuclease, preferably a Cas9 nuclease.

The skilled person knows how to prepare the different components of the CRISPR-CAS system, including CRISPR-nuclease. In the prior art, numerous reports are available on its design and use. See for example the review by Haeussler et al (J Genet Genomics. (2016)43(5):239-50. doi: 10.1016/j.jgg.2016.04.008.) on the design of guide RNA and its combined use with a CAS-protein (originally obtained from S. pyogenes), or the review by Lee et al. (Plant Biotechnology Journal (2016) 14(2) 448-462).

In general, a CRISPR-nuclease, such as Cas9, comprises two catalytically active nuclease domains. For example, a Cas9 protein can comprise a RuvC-like nuclease domain and an HNH-like nuclease domain. The RuvC and HNH domains work together, both cutting a single strand, to make a double-stranded break in DNA. (Jinek et al., Science, 337: 816-821). A dead CRISPR-nuclease comprises modifications such that none of the nuclease domains shows cleavage activity. The CRISPR-nuclease of the gRNA-CAS complex used in the method of the invention may be a variant of a CRISPR-nuclease wherein one of the nuclease domains is mutated such that it is no longer functional (i.e., the nuclease activity is absent), thereby creating a nickase. An example is a SpCas9 variant having either the D10A or H840A mutation. Preferably, the nuclease of the gRNA-CAS complex is not a dead nuclease. Preferably, the CRISPR-nuclease of the gRNA-CAS complex is either a nickase or an (endo)nuclease.

The gRNA-CAS complex that may be used in the method of the invention may comprise or consist of a whole Cas9 protein or variant or may comprise a fragment thereof. Preferably such a fragment does bind crRNA and tracrRNA or sgRNA, and maintains at least one of nuclease or nickase activity.

Preferably, the gRNA-CAS complex comprises a Cas9 protein. The Cas9 protein may be derived from the bacteria Streptococcus pyogenes (SpCas9; NCBI Reference Sequence NC_017053.1; UniProtKB—Q99ZW2), Geobacillus thermodenitrificans (UniProtKB—A0A178TEJ9), Corynebacterium ulcerous (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisl (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); or Neisseria meningitidis (NCBI Ref: YP_002342100.1). Encompassed are Cas9 variants from these, having an inactivated HNH or RuvC domain homologues to SpCas9, e.g. the SpCas9_D10A or SpCas9_H840A, or a Cas9 having equivalent substitutions at positions corresponding to D10 or H840 in the SpCas9 protein, rendering a nickase.

The programmable nuclease may be derived from Cpf1, e.g., Cpf1 from Acidaminococcus sp; UniProtKB—U2UMQ6. The variant may be a Cpf1-nickase having an inactivated RuvC or NUC domain, wherein the RuvC or NUC domain no longer has nuclease activity. The skilled person is well aware of techniques available in the art such as site-directed mutagenesis, PCR-mediated mutagenesis, and total gene synthesis that allow for inactivated nucleases such as inactivated RuvC or NUC domains. An example of a Cpf1 nickase with an inactive NUC domain is Cpf1 R1226A (see Gao et al. Cell Research (2016) 26:901-913, Yamano et al. Cell (2016) 165(4): 949-962). In this variant, there is an arginine to alanine (R1226A) conversion in the NUC-domain, which inactivates the NUC-domain.

The gRNA-CAS complex further comprise a CRISPR-nuclease associated guide RNA that directs the complex to the target sequence or “target site” in the nucleic acid molecule, also annotated as the protospacer sequence. A guide RNA comprises a guide sequence for targeting the gRNA-CAS complex to the protospacer sequence that is preferably near, at or within the sequence of interest in the nucleic acid molecule, and may be a sgRNA or the combination of a crRNA and a tracrRNA (e.g. for Cas9) or a crRNA only (e.g. in case of Cpf1). Optionally, more than one type of guide RNA may be used in the same experiment, for example aimed at two or more different nucleic acid molecules of interest, or even aimed at the same nucleic acid molecule of interest.

In an optional embodiment, the method of the invention is for polymorphism detection and/or detecting genetic variation by using an enzyme that recognizes and cuts heteroduplexes at the site of a mismatch. Within such embodiment, one or more nucleotide samples are fragmented and subsequently undergo at least one round of denaturation and annealing prior or after step b) of the method of the invention. Then, after step c) of the method of the invention, the closed nucleic acids can be treated with the enzyme recognizing and cutting heteroduplexes such as CEL I or an enzyme as described in Langhans MT and Palladino MJ (Curr Issues Mol Biol. 2009; 11(1): 1-12), which is incorporated herein by reference. This results in the opening of only double stranded DNA molecules containing heteroduplexes, which can then be selectively included for further processing (e.g. by ligating sequencing adapters to these open ends and subsequent sequencing) or selectively excluded for further processing (e.g. by degrading these fragments by exonuclease treatment).

In an embodiment, the method may comprise a step e) of exposing the sample to an exonuclease after obtaining the first nucleic acid molecule comprising one open end and one closed end in step d). In this embodiment, the first nucleic acid thus comprises an open end and the second nucleic acid comprises two closed ends. Hence, the second nucleic acid molecule, but not the first nucleic acid molecule, will be protected against exonuclease degradation. Exposure to the exonuclease thus results in digestion of the first nucleic acid, but not the second nucleic acid. In this embodiment, the second nucleic acid preferably comprises a sequence of interest.

The exonuclease may be an exonuclease as defined herein under step c1), optionally under the same or similar conditions as defined herein under step c1). Preferably, exonuclease digestion results in the digestion of all, or substantially all nucleic acid molecules comprising at least one open end. In this embodiment, the method of the invention may therefore comprise the steps of:
a) providing a sample comprising at least a first and a second nucleic acid molecule, wherein the first nucleic acid molecule comprises a first target sequence not present in the second nucleic acid molecule and wherein optionally the second nucleic acid molecule comprises a second target sequence;
b) ligating an adapter as defined herein, i.e. comprising a protelomerase recognition sequence, to the ends of the first and second nucleic acid molecule to provide adapter ligated nucleic acid molecules;
c) contacting the adapter ligated nucleic acid molecules with a protelomerase to cleave and covalently close the cleaved ends, resulting in a first and second nucleic acid molecule comprising closed ends;
d) cleaving the first nucleic acid molecule comprising the closed ends at the first target sequence, to provide a first nucleic acid comprising one open end and one closed end; and
e) exposing the sample to an exonuclease.
   Optionally in between step c) and d), the method may further comprise a step c1) as described herein above.

Optionally, step e) may comprise a step e1) of removing and/or inactivating the restriction endonuclease and/or programmable nuclease, followed by a step e2) of exposing the sample to an exonuclease.

Step e1) may comprise heating the sample to a suitable temperature to remove and/or inactivate the restriction endonuclease and/or programmable nuclease. As a non-limiting example, the temperature may be increased to at least 40° C., 45° C., 50° C., 55° C., 60° C., 65° C., 70° C., 75° C., 80° C. or more. The temperature may be increased for a period of at least about 5′, 10′, 15′, 20′, 25′, 30′, 35′, 40′, 45′, 50′, 55′, 60′ (minutes) or longer.

Alternatively or in addition, step e1) may comprise the purification of the cleaved first nucleic acid molecule. Purification of the cleaved first nucleic acid molecule may be performed using any conventional means, such as, but not limited to an AMPure bead-based purification process and/or partial or complete digestion of the restriction endonuclease and/or programmable nuclease with a proteinase, such as, but not limited to, digestion with proteinase K.

The second nucleic acid molecule comprising two closed ends may subsequently be cleaved at a target sequence. The method of the invention may therefore further comprise a step f) of cleaving the second nucleic acid molecule comprising the closed ends at the second target sequence, resulting in a second nucleic acid comprising one open end and one closed end. The target sequence in the second nucleic acid molecule is preferably not present in the first nucleic acid molecule. However, as within this embodiment the first nucleic acid molecule is already removed at the time the cleaving of the second nucleic acid molecule takes place, optionally the target sequence in the second nucleic acid molecule is also present in the first nucleic acid molecule. Preferably, the method of the invention may comprise the steps of:
a) providing a sample comprising at least a first and a second nucleic acid molecule, wherein the first nucleic acid molecule comprises a first target sequence not present in the second nucleic acid molecule and wherein optionally the second nucleic acid molecule comprises a second target sequence;
b) ligating an adapter as defined herein, i.e. comprising a protelomerase recognition sequence, to the ends of the first and second nucleic acid molecule to provide adapter ligated nucleic acid molecules;
c) contacting the adapter ligated nucleic acid molecules with a protelomerase to cleave and covalently close the cleaved ends, resulting in a first and second nucleic acid molecule comprising closed ends;
d) cleaving the first nucleic acid molecule comprising the closed ends at the first target sequence, to provide a first nucleic acid comprising one open end and one closed end;
e) exposing the sample to an exonuclease; and
f) cleaving the second nucleic acid molecule comprising the closed ends at the second target sequence, resulting in a second nucleic acid comprising one open end and one closed end.
   Optionally in between step c) and d), the method may further comprise a step c1) as described herein above.

Preferably, the second nucleic acid molecule in step f) is cleaved by a programmable nuclease or a restriction endonuclease, preferably a restriction endonuclease as defined in step d) or a programmable nuclease as defined in step d). Preferably, the second nucleic acid molecule in step f) may be digested using a programmable nuclease, preferably using at least one of a CRISPR nuclease, a zinc finger nuclease, TALENs and meganucleases.

Preferably, the second nucleic acid molecule is digested by an RNA-guided CRISPR nuclease. The CRISPR nuclease used for cleaving the first and second nucleic acid molecule may be the same or different. In the case that the CRISPR nucleases used for cleaving the first and second nucleic acid molecules are the same, the guide RNA sequence bound to the CRISPR nuclease is not the same. Put differently, in case CRISPR nucleases are used to cleave the first and second nucleic acid molecule, it is understood herein that the gRNA-Cas complex that recognizes and cleaves the first nucleic acid molecule is a different gRNA-Cas complex that recognizes and cleaves the second nucleic acid molecule.

The method may further comprise a step g) of linking an additional (or “further”) adapter to the open end at least one of the first and second nucleic acid molecule comprising one open and one closed end.

Hence in an embodiment, the method may comprise step a), step b), step c), step d) and step g). Optionally, the method may comprise step a), step b), step c), step c1), step d), and step g). In this embodiment, the additional adapter is linked to the open end of the first nucleic acid molecule. The first nucleic acid molecule preferably comprises a sequence of interest.

In another embodiment, the method may comprise step a), step b), step c), step d), step e), step f) and step g). Optionally, the method may comprise step a), step b), step c), step c1), step d), step e), step f) and step g). In this embodiment, the additional adapter is linked to the open end of the second nucleic acid molecule. The second nucleic acid molecule preferably comprises a sequence of interest.

The additional adapter may be an adapter suitable for amplification and/or sequencing. The additional adapter may be a sequencing adapter, e.g. comprises a functional domain that allows for Roche 454A and 454B sequencing, ILLUMINA™ SOLEXA™ sequencing, Applied Biosystems' SOLID™ sequencing, the Pacific Biosciences' SMRT™ sequencing, Pollonator Polony sequencing, Oxford Nanopore Technologies (ONT), Ontera sequencing or Complete Genomics sequencing.

Hence preferably, the additional adapter comprises at least one sequencing primer binding site and/or the additional adapter comprises at least one amplification primer binding site. The additional adapter may comprise at least two sequencing primer binding sites and/or the further adapter may comprise at least two amplification primer binding site. The additional adapter may be a single-stranded, double-stranded, partly double-stranded, Y-shaped or a hairpin nucleic acid molecule. Preferably, the adapter hairpin adapter or a Y-shaped adapter.

Stem-loop or hairpin adapters are single-stranded, but their termini are complementary such that the adapter folds back on itself to generate a double-stranded portion and a single-stranded loop. A stem-loop adapter can be linked to an end of the linear, double-stranded nucleic acid molecule. For example, where a stem-loop adapter is joined in step g) to the open end of respectively the first or second nucleic acid molecule, there are no terminal nucleotides. The resulting molecule hence lacks terminal nucleotides.

The first or second nucleic acid molecule in step g) may be linked to circularizable adapters. In this respect, nucleic acid molecules comprising an open end may be circularized by self-circularization of compatible structures on either side of the fragment (which may result from adapter ligation or as a result of restriction enzyme digestion of ligated adapters) or circularized by hybridization to a selector probe that is complementary to the ends of the desired fragment. Extension and a final step of ligation creates a covalently closed circular, optionally double-stranded, polynucleotide.

The additional adapter may be a protective adapter. In this context, a protective adapter is to be understood herein as an adapter that is specifically designed to protect the nucleic acid molecule captured by the adapter for exonuclease digestion. Such adapter preferably protects against exonuclease degradation either by the inclusion of chemical moieties or blocking groups (e.g. phosphorothioate) or by a lack of terminal nucleotides (hairpin or stem-loop adapters, or circularizable adapters).

Optionally the additional adapter comprises an identifier sequence, preferably an identifier sequence as defined herein.

Preferably, a nucleic acid molecule library is prepared from a plurality of samples. Optionally, the method of the invention is multiplexed, i.e. applied simultaneously for multiple nucleic acid samples, such as for at least about 10, 20, 30, 40, 50, 60, 70, 80, 90, 100, 500, 1000 or more nucleic acid samples. The method may thus be performed in parallel on a plurality of samples, wherein “in parallel” is to be understood herein as substantially simultaneously but each sample being processed in a separate reaction tube or vessel.

In addition or alternatively, one or more steps of the method of the invention may be performed on pooled samples. In order to trace back the first and/or second nucleic acid molecule to the originating sample, the first and/or second nucleic acid molecule may be tagged with an identifier prior to pooling the samples. Such an identifier can be any detectable entity, such as, but not limited to, a radioactive or fluorescent label, but preferably is a particular nucleotide sequence or combination of nucleotide sequences, preferably of defined length. In addition or alternatively, the samples can be pooled using a clever pooling strategy, such as, but not limited to, a 2D and 3D pooling strategy, such that after pooling each sample is encompassed in at least two or three pools, respectively. A particular nucleic acid molecule can be traced back to the originating sample by using the coordinates of the respective pools comprising the first and/or second nucleic acid molecule. The plurality of samples may be pooled prior to step b) step c), step d) , step e), step f) or prior to step g), or after step g).

In between step a) and b), between step b) and c), between step c) and d) and/or after step d) as described herein, the nucleic sample may be purified and/or the reaction enzyme may be inactivated.

In an embodiment of the invention, between step c) and c1) and/or between step c1) and d) as described herein, the nucleic sample may be purified and/or the reaction enzyme may be inactivated.

In an embodiment of the invention, between step d) and e), between e) and f), between step f) and g), between step d) and g), and/or after step g) as described herein, the nucleic acid sample may be purified and/or the reaction enzyme may be inactivated.

A purification step, e.g., an AMPure bead-based purification process, may be included to remove complexes, enzymes, free nucleotides, possible free adapters, and possible small, non-relevant, nucleic acid molecules. The first, and/or optionally second, nucleic acid molecule may be recovered after purification and subjected to further processing and/or analysis, such as single-molecule sequencing.

An optional purification step is a proteinase K treatment. Alternatively or in addition, said purification may comprise the following steps:
I. exposing the nucleic acid sample to one or more solid supports that specifically and effectively bind the first, and/or optionally second, nucleic acid molecule; and optionally,
II. washing the one or more solid supports and eluting the first, and/or optionally second, nucleic acid molecule from the one or more solid supports.
   The one or more solid supports may be, but not limited to, Ampure beads. As after purification, at least one isolated nucleic acid molecule is obtained, the method as defined herein may also be regarded as a method for isolation of one or more nucleic acid molecules from a nucleic acid sample.

The method of the invention may further comprise a size-selection step. Optionally, the size-selection step is performed prior to step b), between step b) and c), between step c) and d), and/or after step d) of the method of the invention.

In an embodiment, the size selection step is performed in between step c) and c1) and/or between step c1) and d) of the invention.

In an embodiment, the size selection step is performed in between step d) and e), between step e) and f), between step f) and g), or after step g) of the invention.

Alternatively, there is no further purification, inactivation and/or size selection step. Hence in an embodiment, the method of the invention does not require any purification steps between steps a), b), c), d), e), f) and g), or after step g). In addition or alternatively, the method of the invention does not require any inactivation step between steps a), b), c), d), e), f) and g), or after step g). In addition or alternatively, the method of the invention does not require any size selection step between steps a), b), c), d), e), f) and g), or after step g).

The method of the invention may be followed by a step of sequencing one or more target nucleic acid molecules. The method as defined herein may therefore also be also regarded as a method for sequencing one or more target nucleic acid molecules from a nucleic acid sample.

Preferably, the sequencing step is performed after the addition of an adapter comprising a protelomerase recognition sequence. Preferably, the sequencing step is performed after step c), i.e. the sequencing of circular nucleic acid molecule. Preferably, the sequencing step is performed after the addition of a further adapter. Preferably, the sequencing step is performed after step g). Sequencing of at least one of the first and second nucleic acid molecule may be performed after step b), after step c1), after step d), after step e) or after step f).

Optionally, the method of the invention further comprises an amplification step. The amplification step may be performed after closing the nucleic acid molecules comprising an adapter, wherein the adapter comprises a protelomerase recognition sequence. Preferably, the amplification step is performed after step c), i.e. the amplification of a circular nucleic acid molecule. Optionally, the amplification step is performed after annealing a further adapter to the first or second nucleic acid molecule. Preferably, the amplification step is performed after step g). Amplification of at least one of the first and second nucleic acid molecule may be performed after step a), after step b), after step c1), after step d), after step e) and/or after step f). Amplification can be done by PCR or by any amplification method known in the art.

In an embodiment, the method of the invention is a sequencing method that is free of amplification and/or cloning steps. Reduction of amplification steps is beneficial, as epigenetic information (e.g., 5-mC, 6-mA, etc.) will get lost in amplicons. Further amplification can introduce variations in the amplicons (e.g., via errors during amplification) such that their nucleotide sequence is not reflective of the original sample. Similarly, cloning of a target region into another organism often does not maintain modifications present in the original sample nucleic acid, so in some embodiments, target sequences to be enriched for further analysis are typically not amplified and/or cloned in the methods herein.

In an aspect, the method of the invention pertains to a method for amplification of a nucleic acid molecule library. The method preferably comprises a step of preparing nucleic acid molecule library as defined as defined herein. The nucleic acid molecule library is preferably prepared using at least one of:
steps a), b) and c) as defined herein;
steps a), b), c) and c1) as defined herein;
steps a), b), c) and d) as defined herein;
steps a), b), c), c1) and d) as defined herein;
steps a), b), c), d) and g) as defined herein;
steps a), b), c), c1), d) and g) as defined herein;
steps a), b), c), d) and e) as defined herein;
steps a), b), c), c1), d) and e) as defined herein;
steps a), b), c), d), e) and f) as defined herein;
steps a), b), c), c1), d), e) and f) as defined herein;
steps a), b), c), d), e), f) and g) as defined herein; and
steps a), b), c), c1), d), e), f) and g) as defined herein.

The method further comprises a step of amplifying the nucleic acid molecule library. Amplification may be performed using a single primer, e.g. by means of “rolling circle” amplification. The single primer is preferably at least one of:
i) a primer annealing to the first nucleic acid molecule comprising one open and one closed end as obtained in step d);
ii) a primer annealing to the second nucleic acid molecule comprising one open and one closed end as obtained in step f); and
iii) a primer annealing to the further adapter as defined in step g);
   Alternatively or in addition, amplification may be performed using a primer pair, i.e. using a first and a second primer, wherein preferably the first and the second primer can anneal to the first nucleic acid molecule and/or wherein the first and second primer can anneal to the second nucleic acid molecule in a manner that allows for amplification of respectively the first and/or the second nucleic acid molecule.

Preferably, the primer pair comprises a first primer and a second primer that can anneal to the first nucleic acid molecule, preferably the first nucleic acid molecule obtained in step a), b), c), c1), d) or step g) as defined herein. Preferably, the primer pair comprises a first primer and a second primer that can anneal to the first nucleic acid molecule comprising one open and one closed end, as obtained in step d) or step g) as defined herein.

Alternatively or in addition, the primer pair may comprise a first primer and a second primer that can anneal to the second nucleic acid molecule, preferably the second nucleic acid molecule obtained in step a), b), c), c1), d), e), f) or step g) as defined herein. Preferably, the primer pair comprises a first primer and a second primer that can anneal to the second nucleic acid molecule comprising one open and one closed end as obtained in step f) or step g) as defined herein.

Preferably the first primer in the primer pair is not, or not substantially, complementary to the second primer in the primer pair.

In an embodiment, at least one of the first and the second primer may anneal to a sequence present in an adapter, preferably an adapter comprising a protelomerase recognition sequence as defined herein and/or a further adapter as defined in step g).

The first and second primer may anneal to a first sequence and second sequence present in the same adapter, preferably an adapter of step g) as defined herein. As a non-limiting example, the adapter may be a Y-shaped adapter and the first primer binding site may be present in the first single stranded arm of the Y-shaped adapter and the second primer binding site may be present in the other single-stranded arm of the Y-shaped adapter.

Alternatively or in addition, the first amplification primer may anneal to a sequence present in the first nucleic acid molecule and the second amplification primer may anneal to a sequence present in an adapter, preferably an adapter comprising a protelomerase recognition sequence or a further adapter of step g) as defined herein.

Alternatively or in addition, the first amplification primer may anneal to a sequence present in the second nucleic acid molecule and the second amplification primer may anneal to a sequence present in an adapter, preferably an adapter comprising a protelomerase recognition sequence or a further adapter of step g) as defined herein.

Alternatively or in addition, the first amplification primer may anneal to a sequence present in the adapter comprising a protelomerase recognition sequence and the second amplification primer may anneal to a sequence present in the further adapter of step g) as defined herein.

In a further aspect, the invention concerns a method for analysing a sequence of interest in a sample comprising a first and a second nucleic acid molecule. The method preferably comprises a step of preparing a nucleic acid molecule library as defined herein.

The sample may comprise at least a first and a second nucleic acid molecule. The first and/or second nucleic acid molecule may be part of a longer nucleic acid molecule. The nucleic acid sample may comprise a plurality of nucleic acid molecules, including a first and a second nucleic acid molecule.

As detailed herein, the prepared nucleic acid library preferably comprises at least one of a first and a second nucleic acid molecule. In an embodiment, the prepared nucleic acid library comprises a first nucleic acid molecule, but does not comprise the second nucleic acid molecule. In an alternative embodiment, the prepared nucleic acid library comprises a second nucleic acid molecule, but does not comprise the first nucleic acid molecule.

Said first or second nucleic acid molecule preferably comprises a sequence of interest. The nucleic acid molecule library is preferably prepared using at least one of:
steps a), b) and c) as defined herein;
steps a), b), c) and c1) as defined herein;
steps a), b), c), d) and g) as defined herein;
steps a), b), c), c1), d) and g) as defined herein;
steps a), b), c), d), e), f) and g) as defined herein; and
steps a), b), c), c1), d), e), f) and g) as defined herein.

The method preferably further comprises a step of analysing the prepared nucleic acid molecule library. Analysis can be performed using any conventional means known in the art. The analysis may include at least one of:
detection of a sequence using a label, e.g. a radioactive or fluorescent label;
analysis of the size of the prepared nucleic acid molecule library;
cloning, optionally part of, the library into a vector, optionally followed by gene expression and/or restriction analysis; and
sequencing the nucleic acid molecule library.
   Preferably, the prepared nucleic acid molecule library is sequenced, preferably deep-sequenced. Sequencing may include at least one of ILLUMINA™, SOLEXA™ sequencing, Ion Torrent sequencing, the Pacific Biosciences' SMRT™ sequencing, Sanger sequencing, Genapsys, Pollonator Polony sequencing, Oxford Nanopore Technologies (ONT), Ontera sequencing and Complete Genomics sequencing.

In a preferred embodiment, the prepared nucleic acid molecule library is sequenced by nanopore selective sequencing. In nanopore selective sequencing, during real time sequencing the generated data (either direct current signals or base calls translated from these current signals) is compared to one or more reference sequence(s). In case a set number of nucleotides or amount of signals of the target sequence align with the reference sequence, sequencing will proceed, if not, current is reversed thereby removing the nucleic acid from the pore and making the pore available for sequencing of a new nucleic acid. The set number of nucleotides may be at least the first 50, 60, 70, 80, 90, 100, 150, 200, 250, 300, 350, 400, 450, or 500 nucleotides of the nucleic acid read. The one or more reference sequences may be a multitude of different sequences. Preferably, the each of these reference sequences is at least 50, 60, 70, 80, 90, 92, 93, 94, 95, 96, 97 98, 99 or 100% identical to the sequence of a target nucleic acid fragment of the nucleic acid molecule library obtained by the method of the invention. In an embodiment, each of the reference sequences is at least 50, 60, 70, 80, 90, 92, 93, 94, 95, 96, 97 98, 99 or 100% identical to a particular subset of the one or more sequences of target nucleic acid fragments of the nucleic acid molecule library obtained by the method of the invention. One of the benefits of selectively sequencing a particular subset by nanopore selective sequencing is that in different sequencing runs, different subsets may be sequenced using the prepared nucleic acid molecule library.

In an embodiment, the adapter comprising a protelomerase recognition sequence comprises at least one binding site for a sequencing primer.

Alternatively or in addition, the further adapter in step g) comprises at least one binding site for a sequencing primer. The further adapter in step g) may comprise two different binding sites for two sequencing primers. As a non-limiting example, the adapter in step g) may be a Y-shaped adapter and the first sequencing primer binding site may be present in the first single stranded arm of the Y-shaped adapter and the second sequencing primer binding site may be present in the other single-stranded arm of the Y-shaped adapter.

In an aspect, the invention pertains to a method for enriching a nucleic acid sample for a nucleic acid molecule comprising a sequence of interest. The method preferably uses at least method steps a)-d) as detailed herein above, but may use any of the additional steps as detailed herein, such as step c1), step e), step f) and/or step g).

In an aspect, the invention concerns a kit of parts for performing the method of the invention as described herein. Preferably, the kit of parts is for use in a method as defined herein. Preferably, the kit of parts comprises at least one or more adapters comprising a protelomerase recognition sequence as defined herein.

The adapters for use in a method as defined herein preferably do not comprise a recognition site for the restriction endonuclease or the programmable nuclease that is used in step d) and/or step f) of the method of the invention. More preferably the part of the adapter that is located in between the protelomerase recognition sequence and the end ligated to the first and/or second nucleic acid molecule does not comprise a recognition site for a restriction endonuclease or a programmable nuclease that us used in step d) and/or step f) of the method of the invention.

The one or more adapters may be combined in one vial or may be present in separate vials, e.g. wherein the adapters of one vial comprise the same identifier sequence, preferably the same sample identifier sequence. The kit of parts may further comprise a vial comprising a protelomerase as defined herein.

The kit of parts may comprise one or more reagents for performing a method as described herein. Hence the kit of parts may comprise at least one of:
one or more vials comprising adapters comprising a protelomerase recognition sequence as defined herein;
one or more vials comprising a further adapter as defined herein for step g);
one or more vials comprising a protelomerase as defined herein;
one or more vials comprising at a gRNA-CAS complex as defined herein;
one or more vials comprising a gRNA for complexing with a CRISPR-CAS protein to form a gRNA-CAS complex, and a further vial comprising said CRISPR-CAS protein;
a further vial comprising one or more exonucleases.

Preferably, the kit comprises at least 2, 4, 10, 20, 30, or 50 vials comprising one or more gRNAs as defined herein. Preferably, the volume of any of the vials within the kit do not exceed 100 mL, 50 mL, 20 mL, 10 mL, 5 mL, 4 mL, 3 mL, 2 mL or 1 mL.

The reagents may be present in lyophilized form, or in an appropriate buffer. The kit may also contain any other component necessary for carrying out the present invention, such as buffers, pipettes, microtiter plates and written instructions. Such other components for the kits of the invention are known to the skilled person.

In an aspect, the invention pertains to the use of an adapter comprising a protelomerase recognition sequence as defined herein for at least one of:
i) preparation of a nucleic acid molecule library;
ii) amplification of a nucleic acid molecule library; and
iii) analysing a sequence of interest in a sample.

TABLE 1
Description sequence identifiers

| SEQ ID NO | Description |
|---|---|
| 1 | Protelomerase consensus recognition sequence |
| 2 | recognition sequence for E. coli phage NI 5 and |
| | Klebsiella phage phiKO2 protelomerase |
| 3 | recognition sequence for Yersinia phage PY54 |
| | protelomerase |
| 4 | recognition sequence for Halomonas phage phiHAP-1 |
| | protelomerase |
| 5 | recognition sequence for Vibrio phage VP882 |
| 6 | recognition sequence for Borrelia burgdorferi plasmid |
| | lpB31.16 protelomerase |
| 7 | recognition sequence for Vibrio phage VP882 |
| 8 | recognition sequence for Yersinia phage PY54 |
| | protelomerase |
| 9 | Recognition sequence for from Halomonas phage |
| | phiHAP-1 protelomerase |
| 10 | TelN protelomerase recognition sequence |
| 11 | Amino acid sequence protelomerase |
| 12 | Cas9 amino acid sequence |
| 13 | Cas9 nucleotide sequence |
| 14 | Cas9 amino acid sequence |
| 15 | Cpf1 amino acid sequence |
| 16 | Cpf1 nucleotide sequence |
| 17 | MAD7 protein sequence |
| 18 | MAD7 protein sequence |

### Figure Legend

FIG. 1: Agilent 2100 Bioanalyzer DNA Analysis using the DNA 12000 kit. On the left side of the boxes the DNA size marker including the fragment lengths are indicated. The left box shows the amplicon (˜1050 bp) which is used as input in the experiment, without (left) and with (right) exonuclease (ExoV) treatment. In the middle box the amplicon to which a TeIN adapter is ligated is shown, without (left) and with (right) ExoV treatment. The right box shows the amplicon with ligated TeIN adapters treated with TeIN protelomerase, with (left) and without (right) ExoV treatment. Results show that TeIN treatment of the adapter ligated amplicon results in protection from ExoV degradation.

### EXAMPLES

### Material and Methods

An adapter containing the TeIN recognition site was prepared by combining:

### Oligo 19_04626 (100 μM): 2 μl

### Oligo 19_03053 (100 μM): 2 μl

Sequences of the oligos:

19_04626
(SEQ ID NO: 19)
5′-AGGACCGGATCAACTTATCAGCACACAATTGCCCATTATACGCGC
GTATAATGGACTATTGTGTGCTGATAAAGAAAGTTGTCGGTGTCTTTG
TGAGATGTGTATAAGAGACAGT-3′
19_03053
(SEQ ID NO: 20)
5′-CTGTCTCTTATACACATCTCACAAAGACACCGACAACTTTCTTTA
TCAGCACACAATAGTCCATTATACGCGCGTATAATGGGCAATTGTGTG
CTGATAAGTTGATCCGGTCCT-3′

The 5′-end is preferably phosphorylated.
To enable hybridization of the oligos the following thermal profile was used:

### 95° C. 10 min

### 90° C. for 1 min

Reduce temp by 1° C./cycle 60 times
4° C. hold
The resulting adapter solution (50 μM) was diluted to 15 μM concentration.
Input material for the example was a 1 Kbp amplicon derived from Lambda DNA.
The amplification was performed using the following setup:

| | | |
|---|---|---|
| Lambda DNA 5 ng/μl | 5 | μl |
| MilliQ water | 9.3 | μl |
| PCR buffer | 4 | μl |
| 25 mM dNTP (each) | 0.2 | μl |
| Herculase Polymerase | 0.5 | μl |
| Forward primer (10 μM) | 0.5 | μl |
| Reverse primer (10 μM) | 0.5 | μl |

Forward primer: 18_03029:
(SEQ ID NO: 21)
5′-TCACGCTGATTTACAGCGGCA-3′
Reverse primer: 18_03032:
(SEQ ID NO: 22)
5′-CGATGCTGATTGCCGTTCCG-3′

For amplification the thermoprofile used was:

### 95° C. 2 min

### 95° C. for 30 sec

65° C. for 30 sec -> reducing temp by 0.7° C./cycle

### 72° C. for 4 min

13 cycles

### 95° C. for 30 sec

### 56° C. for 30 sec

### 72° C. for 5 min

25 cycles

### 72° C. for 2 min

12° C. hold
The resulting amplicon was 0.8× purified and eluted in 20 ul MQ. The concentration was measured at the Qubit BR: 554 ng/μl
The purified amplicon was end repaired and A-tailed.
End repair (two reactions performed):
2 μl of purified amplicon

### 7 μl NEBNext Ultra II End Prep Reaction Buffer (New England Biolabs Inc.)

### 3 μl NEBNext Ultra II End Prep Enzyme Mix (New England Biolabs Inc.)

48 μl MilliQ water
Total volume=60 μl -> incubated for 30 min 20° C., 30 min 65° C. and hold at 4° C. until further use.
Adapter ligation:

### 60 μl NEBNext Ultra II End Prep Reaction Mixture (New England Biolabs Inc.)

### 30 μl NEBNext Ultra II Ligation Master Mix (New England Biolabs Inc.)

### 1 μl NEBNext Ligation Enhancer (New England Biolabs Inc.)

### 2.5 μl Adapter (50 μM)

Total volume=93.5 μl -> incubated for 20 min 15° C.
The resulting ligated sample was purified using 1:1 Ampure beads and eluted in 20 μl MilliQ water.
To remove remaining adapters, an additional Ampure purification (0.75×) was performed.
Concentration of the adapter ligated product is 40 ng/μl
The adapter ligated product was treated with TeIN to covalently close the ends.

| | | |
|---|---|---|
| Adapter ligated product | 4 | μl |
| ThermoPol Reaction Buffer (10x) (New England Biolabs Inc.) | 2 | μl |
| TeIN Protelomerase (New England Biolabs Inc.) | 2 | μl |
| MilliQ water | 12 | μl |

The reaction mixture was gently mixed by pipetting up and down, briefly centrifuged and incubated at 30° C. for 30 min. The enzyme was inactivated by incubating at 75° C. for 5 min.
The resulting sample was purified using 1:1 Ampure beads and eluted in 15 μl MilliQ water.
To verify exonuclease protection, the TeIN treated sample was incubated with Exonuclease V.

| | | |
|---|---|---|
| Sample | 10 | μl |
| NEB Buffer 3.1 (10x) | 2.0 | μl |
| ATP (100 mM) | 1.0 | μl |
| Exonuclease V (10 units) | 1.0 | μl |
| MilliQ water | 6.0 | μl |

The reaction mixture was incubated at 37° C. for 60 min. and the Exonuclease was inactivated at 70° C. for 30 min.
The sample was purified using Ampure (1×) and eluted in 10 ul MilliQ water.

### Results

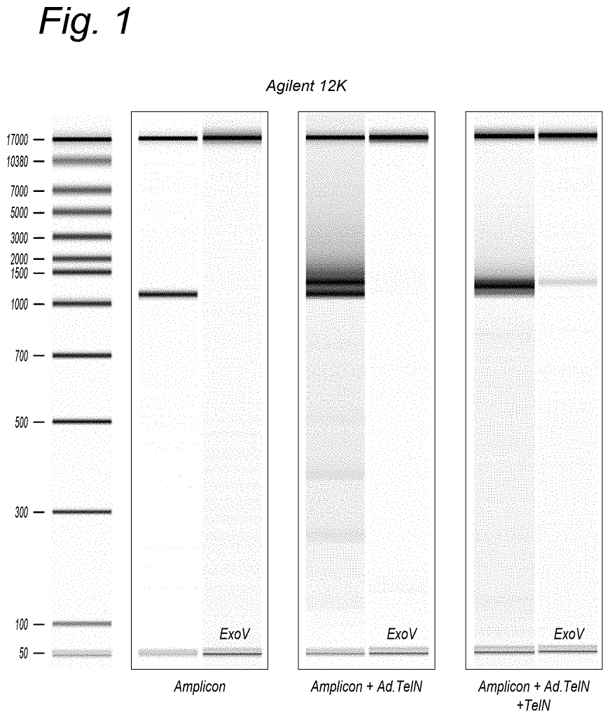

Results of the Bioanalyzer analysis are shown in FIG. 1: In brief;
Amplicons and adapter-ligated amplicons are readily degraded using Exonuclease V Adapter-ligated and TeIN treated amplicons are resistant to Exonuclease degradation

### Conclusion

Covalently closing the ends of DNA fragments using TeIN, renders ExonucleaseV resistant fragments.

1. An adapter comprising at least partly a double-stranded nucleic acid and a protelomerase recognition sequence.

2. The adapter according to claim 1, wherein the protelomerase recognition sequence is a TeIN protelomerase recognition sequence.

3. The adapter according to claim 1, further comprising an identifier sequence.

4. The adapter according to claim 1, wherein the adapter comprises at least one staggered end.

5. A method for preparing a nucleic acid molecule library, the method comprising:
(a) providing a sample comprising at least a first and a second nucleic acid molecule, wherein the first nucleic acid molecule comprises a first target sequence not present in the second nucleic acid molecule and wherein optionally the second nucleic acid molecule comprises a second target sequence;
(b) ligating an adapter according to claim 1 to the ends of the first and second nucleic acid molecules to provide adapter ligated nucleic acid molecules;
(c) contacting the adapter ligated nucleic acid molecules with a protelomerase to cleave and covalently close the cleaved ends, resulting in a first and second nucleic acid molecule comprising closed ends; and
(d) cleaving the first nucleic acid molecule comprising the closed ends at the first target sequence, to provide a first nucleic acid comprising one open end and one closed end.

6. The method according to claim 1, wherein the protelomerase recognition sequence is a TeIN protelomerase recognition sequence.

7. The method according to claim 5, wherein the sample further comprises a plurality of additional nucleic acid molecules.

8. The method according to claim 5, wherein the first nucleic acid molecule is cleaved by a programmable nuclease or a restriction endonuclease.

9. The method according to claim 8, wherein the programmable nuclease is an RNA-guided CRISPR nuclease.

10. The method according to claim 5, wherein the first and second nucleic acid molecules are provided by fragmentation of a genomic nucleic acid molecule.

11. The method according to claim 5, wherein the adapter is ligated by tagmentation.

12. The method according to claim 5, wherein the method further comprises (c1) exposing the sample to an exonuclease, after obtaining the nucleic acid molecules comprising closed ends and prior to cleaving the first nucleic acid molecule comprising the closed ends.

13. The method according to claim 5, wherein the method further comprises (e) exposing the sample to an exonuclease after obtaining the first nucleic acid molecule comprising one open end and one closed end.

14. The method according to claim 13, wherein the method further comprises (f) cleaving the second nucleic acid molecule comprising the closed ends at the second target sequence, resulting in a second nucleic acid comprising one open end and one closed end.

15. The method according to claim 5, wherein the method further comprises (g) linking a further adapter to the open end of the first, or optionally second, nucleic acid molecule comprising one open and one closed end, wherein the further adapter comprises at least one of an amplification primer binding site and sequence primer binding site and optionally an identifier sequence.

16. The method according to claim 5, wherein a nucleic acid molecule library is prepared from a plurality of samples and pooled.

17. The method according to claim 6, wherein the adapter ligated nucleic acid molecules are repaired to remove single-stranded breaks prior to contacting the molecules with a TeIN protelomerase.

18. A method for amplification of a nucleic acid molecule library, the method comprising:
(a) preparing a nucleic acid molecule library according to claim 5; and
(b) amplifying the nucleic acid molecule library using at least one of:
(i) a first, and optionally second, primer annealing to the first nucleic acid molecule comprising one open and one closed end;
(ii) a first, and optionally a second, primer annealing to the second nucleic acid molecule comprising one open and one closed end;
(iii) a first, and optionally a second, primer annealing to the further adapter; and
(iv) a combination of a first primer as defined in (i) or (ii), and a second primer as defined in (iii).

19. A method for analysing a sequence of interest in a sample comprising a first and a second nucleic acid molecule, comprising:
(a) preparing a nucleic acid molecule library according to claim 5;
(b) optionally amplifying the prepared nucleic acid molecule library; and
(c) sequencing the nucleic acid molecule library.

20. The method according to claim 19, wherein the sequencing comprises deep-sequencing.

21. A kit of parts comprising:
(a) one or more adapters according to claim 1; and
(b) optionally a protelomerase.
